(19) United States
(12) Patent Application Publication
(10) Pub. No.: US 2011/0256130 A1
(43) Pub. Date: Oct. 20, 2011

(54) METHODS OF TREATING INFLAMMATORY DISORDERS

(76) Inventors: Joshua Robert Schultz, Ballston Lake, NY (US); Benedikt Vollrath, San Diego, CA (US); Court Turner, San Diego, CA (US)

(21) Appl. No.: 13/125,035

(22) PCT Filed: Dec. 1, 2009
(86) PCT No.: PCT/US2009/066180

§ 371 Date: Jun. 23, 2011

Related U.S. Application Data
(60) Provisional application No. 61/118,938, filed on Dec. 1, 2008.
(60) Provisional application No. 61/121,779, filed on Dec. 11, 2008.

(51) Int. Cl. A61K 39/395; A61P 7/06; A61P 3/10; A61P 25/00; A61P 3/04; A61P 9/00; A61P 29/00; A61P 25/18; A61P 17/00; A61P 35/00; A61P 11/06; A61P 25/28; A61P 11/00; A61P 27/02; A61P 37/06; A61P 3/00; A61P 1/16; A61P 1/00; A61P 19/02; A61P 25/08; A61P 25/16; A61P 17/06; A61P 1/18; A61K 38/10

(52) U.S. Cl. 424/133.1; 514/21.5; 514/13.5; 514/6.9; 514/7.3; 514/17.9; 514/4.8; 514/16.4; 514/16.6; 514/17.5; 514/18.6; 514/19.3; 514/19.5; 514/17.7; 514/1.7; 514/17.8; 514/1.5; 514/1.8; 514/20.8

(57) ABSTRACT

Disclosed herein, in certain embodiments, are methods and compositions for treating inflammatory disorders. In some embodiments, the methods comprise co-administering synergistic combinations of modulators of inflammation.

### CROSS-REFERENCE

This application claims the benefit of U.S. Provisional Application No. 61/118,938, filed Dec. 1, 2008, and U.S. Provisional Application No. 61/121,779, filed Dec. 11, 2008 both of which are incorporated herein by reference.

### BACKGROUND OF THE INVENTION

Certain inflammatory conditions are characterized, in part, by the migration of lymphocytes into the effected tissue. The migration of lymphocytes induces tissue damage and exacerbates inflammatory conditions.

### SUMMARY OF THE INVENTION

We recognize that there is a need to develop methods and compositions for treating inflammatory disorders that combine (a) an agent that inhibits inflammation through the modulation of a first pathway with (b) an agent that inhibits inflammation through the modulation of a second pathway.

We further recognize that in certain instances, agents that treat inflammatory disorders (e.g., Rheumatoid Arthritis (RA), Systemic Lupus Erythmateous (SLE)) have side effects or adverse aspects associated with undesired inflammation (e.g., the anti-inflammatory initiates an inflammatory response by killing cells; initiating the expression of chemokines; initiating the expression of cytokines; depleting intracellular cholesterol, interfering with oxidative phosphorylation pathways; activating natural killer cells, mast cells, eosinophils, basophils; macrophages, neutrophils and/or dendritic cells; and/or activating the classic complement cascade and/or the alternative complement cascade). As a result, such agents are abandoned or administered in amounts that limit such side effects or adverse aspects even, in some cases, where a higher dose of such agents would otherwise provide benefit in the treatment of the inflammatory disorder. In other instances, the use of such agents is discontinued if the individual experiences or is expected to experience such side effects or adverse events. In yet certain other instances, the benefit that the agent provides to one aspect of the inflammatory disorder in counteracted, at least in part, from the inflammatory aspects of the agent.

Further disclosed herein, in certain embodiments, are methods and compositions for reducing or preventing inflammation in a patient that has an inflammatory disorder and is under prescription for a therapeutic agent that either (i) inhibits inflammation and modulates a lipid, or (ii) induces unwanted inflammation, the present compositions comprising a modulator of MIF, and the methods comprising administering to the patient a synergistic or inflammation-reducing amount of such an active agent.

Disclosed herein, in certain embodiments, are methods and pharmaceutical compositions for modulating an inflammatory disorder comprising a synergistic combination of a modulator of MIF; and a second active agent that treats inflammation through an alternative pathway.

Further disclosed herein, in certain embodiments, are methods and compositions for treating inflammatory disorders. In some embodiments, the method comprises co-administering a synergistic combination of (a) a modulator of MIF; and (b) a second active agent selected from an agent that inhibits inflammation and modulates a lipid.

Further disclosed herein, in certain embodiments, are methods and compositions for treating inflammatory disorders. In some embodiments, the method comprises co-administering a synergistic combination of (a) a modulator of MIF; and (b) a second active agent selected from an agent that induces unwanted inflammation.

In some embodiments, the combination is synergistic and results in a more efficacious therapy. In some embodiments, therapy synergistically treats inflammatory disorders by targeting multiple pathways that result in (either partially or fully) development of an inflammatory disorder.

In some embodiments, the modulator of MIF and a fibrate synergistically treat an inflammatory disorder by (1) decreasing the chemotaxis of leukocytes, and (2) increasing the concentration of HDL. In some embodiments, the first active agent also decreases any undesired inflammation resulting from administration of the fibrate.

In some embodiments, the modulator of MIF and an ApoA1 modulator synergistically treat an inflammatory disorder by (1) decreasing the chemotaxis of leukocytes, and (2) increasing the concentration of HDL. In some embodiments, the first active agent also decreases any undesired inflammation resulting from administration of the ApoA1 modulator.

In some embodiments, the modulator of MIF and a CETP modulator synergistically treat an inflammatory disorder by (1) decreasing the chemotaxis of leukocytes, and (2) increasing the concentration of HDL. In some embodiments, the first active agent also decreases any undesired inflammation resulting from administration of the CETP inhibitor.

In some embodiments, the modulator of MIF and an anti-TNF agent treat an inflammatory disorder by (1) decreasing the chemotaxis of leukocytes, and (2) suppressing a TNF-induced cytokine cascade. In some embodiments, the first active agent also decreases any undesired inflammation (e.g., tuberculosis) resulting from administration of the anti-TNF agent.

In some embodiments, the modulator of MIF and an IL-1 receptor antagonist treat an inflammatory disorder by (1) decreasing the chemotaxis of leukocytes, and (2) blocking the stimulation of T cell IL-1 receptor. In some embodiments, the first active agent also decreases any undesired inflammation (e.g., pneumonia, and bone and joint infections) resulting from administration of the IL-1 receptor antagonist.

In some embodiments, the modulator of MIF and an IL-2 receptor antagonist treat an inflammatory disorder by (1) decreasing the chemotaxis of leukocytes, and (2) blocking the stimulation of T cell IL-2 receptor. In some embodiments, the first active agent also decreases any undesired inflammation (e.g., gastrointestinal disorders) resulting from administration of the IL-2 receptor antagonist.

In some embodiments, the modulator of MIF and a cytotoxic agent treat an inflammatory disorder by (1) decreasing the chemotaxis of leukocytes, and (2) treating neoplastic disease. In some embodiments, the first active agent also decreases any undesired inflammation (e.g., neutropenia) resulting from administration of the cytotoxic agent.

In some embodiments, the modulator of MIF and an immunomodulatory agent treat an inflammatory disorder by (1) decreasing the chemotaxis of leukocytes, and (2) enhancing, or suppressing the immune system. In some embodiments, the first active agent also decreases any undesired inflammation (e.g., hematologic side effects) resulting from administration of the immunomodulatory agent.

In some embodiments, the modulator of MIF and an antibiotic treat an inflammatory disorder by (1) decreasing the chemotaxis of leukocytes, and (2) by blocking cell and/or microbial growth by disrupting the cell cycle, or by blocking histone deacetylase. In some embodiments, the first active agent also decreases any undesired inflammation (e.g., cardiotoxicity) resulting from administration of the antibiotic.

In some embodiments, the modulator of MIF and a T-cell co-stimulatory blocker treat an inflammatory disorder by (1) decreasing the chemotaxis of leukocytes, and (2) modulating a co-stimulatory signal which is required for full T-cell activation. In some embodiments, the first active agent also decreases any undesired inflammation (e.g., neutropenia) resulting from administration of the T-cell co-stimulatory blocker.

In some embodiments, the modulator of MIF and a B cell depleting agent treat an inflammatory disorder by (1) decreasing the chemotaxis of leukocytes, and (2) inhibiting B-cell activity. In some embodiments, the first active agent also decreases any undesired inflammation (e.g., Progressive Multifocal Leukoencephalopathy) resulting from administration of the B-cell depleting agent.

In some embodiments, the modulator of MIF and an immunosuppressive agent treat an inflammatory disorder by (1) decreasing the chemotaxis of leukocytes, and (2) selectively or non-selectively inhibiting and/or preventing activity of the immune system. In some embodiments, the first active agent also decreases any undesired inflammation (e.g., lymphoma) resulting from administration of immunosuppressive agent.

In some embodiments, the modulator of MIF and an alkylating agent treat an inflammatory disorder by (1) decreasing the chemotaxis of leukocytes, and (2) inducing covalent binding of alkyl groups to cellular molecules. In some embodiments, the first active agent also decreases any undesired inflammation (e.g., immune suppression) resulting from administration of the alkylating agent.

In some embodiments, the modulator of MIF and an anti-metabolite treat an inflammatory disorder by (1) decreasing the chemotaxis of leukocytes, and (2) preventing the biosynthesis or use of normal cellular metabolites. In some embodiments, the first active agent also decreases any undesired inflammation (e.g., mutagenesis) resulting from administration of the anti metabolite.

In some embodiments, the modulator of MIF and a plant alkaloid treat an inflammatory disorder by (1) decreasing the chemotaxis of leukocytes, and (2) interfering with normal microtubule breakdown during cell division. In some embodiments, the first active agent also decreases any undesired inflammation (e.g., leukopenia) resulting from administration of the plant alkaloid.

In some embodiments, the modulator of MIF and a terpenoid treat an inflammatory disorder by (1) decreasing the chemotaxis of leukocytes, and (2) treating neoplastic disease or microbial infections. In some embodiments, the first active agent also decreases any undesired inflammation resulting from administration of the terpenoid agent.

In some embodiments, the modulator of MIF and a topoisomerase inhibitor treat an inflammatory disorder by (1) decreasing the chemotaxis of leukocytes, and (2) modulating the action of cellular topoisomerase enzymes. In some embodiments, the first active agent also decreases any undesired inflammation (e.g., gastrointestinal effects) resulting from administration of the topoisomerase inhibitor.

In some embodiments, the modulator of MIF and an antibody treat an inflammatory disorder by (1) decreasing the chemotaxis of leukocytes, and (2) neutralizing inflammatory cytokines such as, for example, TNF alpha. In some embodiments, the first active agent also decreases any undesired inflammation (e.g., tuberculosis) resulting from administration of the antibody.

In some embodiments, the modulator of MIF and a hormonal therapy treat an inflammatory disorder by (1) decreasing the chemotaxis of leukocytes, and (2) suppressing cytokine release. In some embodiments, the first active agent also decreases any undesired inflammation (e.g., cancer) resulting from administration of the hormone.

In some embodiments, the modulator of MIF and an anti-diabetes therapy treat an inflammatory disorder by (1) decreasing the chemotaxis of leukocytes, and (2) improving sensitivity to insulin in muscle and adipose tissue. In some embodiments, the first active agent also decreases any undesired inflammation (e.g., liver inflammation, pancreatitis) resulting from administration of the anti-diabetes agent.

In some embodiments, the modulator of MIF and a leukotriene inhibitor treat an inflammatory disorder by (1) decreasing the chemotaxis of leukocytes, and (2) antagonizing LTA4, LTB4, LTC4, LTD4, LTE4, LTF4, LTA4R; LTB4R; LTB4R1, LTB4R2, LTC4R, LTD4R, LTE4R, CYSLTR1, or CYSLTR2; or inhibiting the synthesis of a leukotriene via 5-LO, FLAP, LTA4H, LTA4S, or LTC4S. In some embodiments, the first active agent also decreases any undesired inflammation (e.g., tuberculosis) resulting from administration of the leukotriene inhibitor.

Disclosed herein, in certain embodiments, is a method of treating an inflammatory disorder in an individual in need thereof, comprising administering to the individual a synergistic combination of (a) a therapeutically-effective amount of a modulator of MIF; and (b) a second active agent selected from an agent that modulates a lipid and/or a lipoprotein. In some embodiments, the second active agent modulates the concentration of HDL in an individual. In some embodiments, the second active agent is a fibrate; an apolipoprotein A-1 modulator; a CETP modulator; or combinations thereof. In some embodiments, the modulator of MIF inhibits (i) MIF binding to CXCR2 and CXCR4; (ii) MIF-activation of CXCR2 and CXCR4; (iii) the ability of MIF to form a homomultimer; or a combination thereof. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with an N-Loop motif of MIF. In some embodiments, the modulator of MIF inhibits binding of an N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combinations thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N-(2,3-dichlorophenyl) urea, IL-8(1-72), (R)IL-8, (R)IL-8, NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, the modulator of MIF is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, the administration of the second active agent partially or fully results in undesired inflammation. In some embodiments, the administration of the second active agent partially or fully results in inflammation. In some embodiments, the modulator of MIF treats and/or ameliorates the inflammation induced by administration of the second active agent. In some embodiments, co-administering the modulator of MIF with the second active agent rescues the individual from inflammation induced by administration of the second active agent. In some embodiments, the second active agent is bezafibrate; ciprofibrate; clofibrate; gemfibrozil; fenofibrate; or combinations thereof. In some embodiments, the second active agent is DF4 (Ac-D-W-F-K-A-F-Y-D-K-V-A-E-K-F-K-E-A-F—NH2); DF5; RVX-208 (Resverlogix); or combinations thereof. In some embodiments, the second active agent is torcetrapib; anacetrapid; JTT-705 (Japan Tobacco/Roche); or combinations thereof. In some embodiments, the inflammatory disorder partially or fully results from obesity, metabolic syndrome, an immune disorder, an Neoplasm, an infectious disorder, a chemical agent, an inflammatory bowel disorder, reperfusion injury, necrosis, or combinations thereof. In some embodiments, the inflammatory disorder is an autoimmune disorder, an allergy, a leukocyte defect, graft versus host disease, tissue transplant rejection, or combinations thereof. In some embodiments, the inflammatory disorder is a bacterial infection, a protozoal infection, a viral infection, a fungal infection, or combinations thereof. In some embodiments, the inflammatory disorder is Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjë gren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, abdominal aortic aneurysm (AAA), or combinations thereof.

Disclosed herein, in certain embodiments, is a method of treating an inflammatory disorder in an individual in need thereof, comprising administering to the individual a synergistic combination of (a) a therapeutically-effective amount of a modulator of MIF; and (b) a second active agent selected from an anti-inflammatory agent. In some embodiments, the second active agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent an alkylating agent, an anti-metabolite, a plant alkaloid, a terpenoids, a topoisomerase inhibitor, an antitumour antibiotic, a antibody, a hormonal therapy, an anti-diabetes agent, a leukotriene inhibitor, or combinations thereof. In some embodiments, the second active agent is alefacept, efalizumab, methotrexate, acitretin, isotretinoin, hydroxyurea, mycophenolate mofetil, sulfasalazine, 6-Thioguanine (thioguanine, 6-TG, 2-Amino-6-Mercaptopurine), Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), ), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); doxycycline; or combinations thereof. In some embodiments, the inflammatory disorder partially or fully results from obesity, metabolic syndrome, an immune disorder, an Neoplasm, an infectious disorder, a chemical agent, an inflammatory bowel disorder, reperfusion injury, necrosis, or combinations thereof. In some embodiments, the inflammatory disorder is an autoimmune disorder, an allergy, a leukocyte defect, graft versus host disease, tissue transplant rejection, or combinations thereof. In some embodiments, the inflammatory disorder is a bacterial infection, a protozoal infection, a protozoal infection, a viral infection, a fungal infection, or combinations thereof. In some embodiments, the inflammatory disorder is Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjë gren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, abdominal aortic aneurysm (AAA); or combinations thereof. In some embodiments, the modulator of MIF inhibits (i) MIF binding to CXCR2 and CXCR4; (ii) MIF-activation of CXCR2 and CXCR4; (iii) the ability of MIF to form a homomultimer; or a combination thereof. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with an N-Loop motif of MIF. In some embodiments, the modulator of MIF inhibits binding of an N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combinations thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N′-(2,3-dichlorophenyl) urea, IL-8(1-72), (R)IL-8, (R)IL-8, NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, the modulator of MIF is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, the administration of the second active agent partially or fully results in undesired inflammation. In some embodiments, the administration of the second active agent partially or fully results in inflammation. In some embodiments, the modulator of MIF treats and/or ameliorates the inflammation induced by administration of the second active agent. In some embodiments, co-administering the modulator of MIF with the second active agent rescues the individual from inflammation induced by administration of the second active agent.

Disclosed herein, in certain embodiments, is a pharmaceutical composition for modulating an inflammation, comprising a synergistic combination of a (a) therapeutically-effective amount of a modulator of MIF; and (b) therapeutically-effective amount of a second active agent selected from a modulator of a lipid disorder. In some embodiments, the modulator of MIF inhibits (i) MIF binding to CXCR2 and CXCR4; (ii) MIF-activation of CXCR2 and CXCR4; (iii) the ability of MIF to form a homomultimer; or a combination thereof. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with an N-Loop motif of MIF. In some embodiments, the modulator of MIF inhibits binding of an N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combinations thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N-(2,3-dichlorophenyl) urea, IL-8(1-72), (R)IL-8, (R)IL-8, NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, the modulator of MIF is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, the administration of the second active agent partially or fully results in undesired inflammation. In some embodiments, the administration of the second active agent partially or fully results in inflammation. In some embodiments, the modulator of MIF treats and/or ameliorates the inflammation induced by administration of the second active agent. In some embodiments, co-administering the modulator of MIF with the second active agent rescues the individual from inflammation induced by administration of the second active agent. In some embodiments, the second active agent is a fibrate; an apolipoprotein A-1 modulator; a CETP modulator; or combinations thereof. In some embodiments, the second active agent is bezafibrate; ciprofibrate; clofibrate; gemfibrozil; fenofibrate; or combinations thereof. In some embodiments, the second active agent is DF4 (Ac-D-W-F-K-A-F-Y-D-K-V-A-E-K-F-K-E-A-F—NH2); DF5; RVX-208 (Resverlogix); or combinations thereof. In some embodiments, the second active agent is torcetrapib; anacetrapid; JTT-705 (Japan Tobacco/Roche); or combinations thereof.

Disclosed herein, in certain embodiments, is a pharmaceutical composition for modulating an inflammation, comprising a synergistic combination of (a) a therapeutically-effective amount of a a modulator of MIF; and (b) a therapeutically-effective amount of a second active agent selected from an anti-inflammatory. In some embodiments, the modulator of MIF inhibits (i) MIF binding to CXCR2 and CXCR4; (ii) MIF-activation of CXCR2 and CXCR4; (iii) the ability of MIF to form a homomultimer; or a combination thereof. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with an N-Loop motif of MIF. In some embodiments, the modulator of MIF inhibits binding of an N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combinations thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N-(2,3-dichlorophenyl) urea, IL-8(1-72), (R)IL-8, (R)IL-8, NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, the modulator of MIF is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, the administration of the second active agent partially or fully results in inflammation. In some embodiments, the modulator of MIF treats and/or ameliorates the inflammation induced by administration of the second active agent. In some embodiments, co-administering the modulator of MIF with the second active agent rescues the individual from inflammation induced by administration of the second active agent. In some embodiments, the second active agent is an anti-inflammatory agent. In some embodiments, the second active agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent, an alkylating agent, an anti-metabolite, a plant alkaloid, a terpenoids, a topoisomerase inhibitor, an antitumour antibiotic, an antibody, a hormonal therapy, an anti-diabetes agent, a leukotriene inhibitor, or combinations thereof. In some embodiments, the second active agent is alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), ), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol, Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S—[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); doxycycline; or combinations thereof. In some embodiments, the composition comprises a first population of particles and a second population of particles. In some embodiments, the first population of particles is formulated for immediate release. In some embodiments, the second population of particles is formulated for controlled release. In some embodiments, the first population of particles comprises a therapeutically-effective amount of an agent that treats an inflammatory disorder. In some embodiments, the second population of particles comprises a therapeutically-effective amount of a modulator of MIF.

### DETAILED DESCRIPTION OF THE INVENTION

Disclosed herein, in certain embodiments, are methods and pharmaceutical compositions for modulating an inflammatory disorder comprising a synergistic combination of (a) a modulator of MIF; and (b) a second active agent that treats inflammation through an alternative pathway.

Further disclosed herein, in certain embodiments, are methods and compositions for treating inflammatory disorders. In some embodiments, the method comprises co-administering a synergistic combination of (a) a modulator of MIF; and (b) a second active agent selected from an agent that inhibits inflammation and modulates a lipid.

In some embodiments, the combination is synergistic and results in a more efficacious therapy. In some embodiments, therapy synergistically treats inflammatory disorders by targeting multiple pathways that result in (either partially or fully) development of an inflammatory disorder.

In some embodiments, the modulator of MIF and a fibrate synergistically treat an inflammatory disorder by (1) decreasing the chemotaxis of leukocytes, and (2) increasing the concentration of HDL. In some embodiments, the modulator of MIF also decreases any undesired inflammation resulting from administration of the fibrate.

In some embodiments, the modulator of MIF and an ApoA1 modulator synergistically treat an inflammatory disorder by (1) decreasing the chemotaxis of leukocytes, and (2) increasing the concentration of HDL. In some embodiments, the modulator of MIF also decreases any undesired inflammation resulting from administration of the ApoA1 modulator.

In some embodiments, the modulator of MIF and a CETP modulator synergistically treat an inflammatory disorder by (1) decreasing the chemotaxis of leukocytes, and (2) increasing the concentration of HDL. In some embodiments, the modulator of MIF also decreases any undesired inflammation resulting from administration of the CETP inhibitor.

### I. DEFINITIONS

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. In some embodiments, the mammal is an Non-human. In some embodiments, the mammal is a member of the taxonomic orders: primates (e.g. lemurs, lorids, galagos, tarsiers, monkeys, apes, and humans); rodentia (e.g. mice, rats, squirrels, chipmunks, and gophers); lagomorpha (e.g. hares, rabbits, and pika); erinaceomorpha (e.g. hedgehogs and gymnures); soricomorpha (e.g. shrews, moles, and solenodons); chiroptera (e.g., bats); cetacea (e.g. whales, dolphins, and porpoises); carnivora (e.g. cats, lions, and other feliformia; dogs, bears, weasels, and seals); perissodactyla (e.g. horse, zebra, tapir, and rhinoceros); artiodactyla (e.g. pigs, camels, cattle, and deer); proboscidea (e.g. elephants); sirenia (e.g. manatees, dugong, and sea cows); cingulata (e.g. armadillos); pilosa (e.g. anteaters and sloths); didelphimorphia (e.g. american opossums); paucituberculata (e.g. shrew opossums); microbiotheria (e.g. Monito del Monte); notoryctemorphia (e.g. marsupial moles); dasyuromorphia (e.g. marsupial carnivores); peramelemorphia (e.g. bandicoots and bilbies); or diprotodontia (e.g. wombats, koalas, possums, gliders, kangaroos, wallaroos, and wallabies). In some embodiments, the animal is a reptile (i.e. species of any orders, families, and genus within the taxonomic classification animalia: chordata: vertebrata: reptilia). In some embodiments, the animal is a bird (i.e. animalia: chordata: vertebrata: ayes). None of the terms require or are limited to situation characterized by the supervision (e.g. constant or intermittent) of a health care worker (e.g. a doctor, a registered nurse, an Nurse practitioner, a physician's assistant, an orderly, or a hospice worker).

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to an individual simultaneously in the form of a single entity or dosage. The term “non-fixed combination” means that at least one of the agents described herein, and at least one co-agent, are administered to an individual as separate entities either simultaneously, concurrently or sequentially with variable intervening time limits, wherein such administration provides effective levels of the two or more agents in the body of the individual. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single individual, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the agents and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, a agent of this invention or a pharmaceutically active metabolite or residue thereof. Particularly favored prodrugs are those that increase the bioavailability of the agents of this invention when such agents are administered to an individual (e.g., by allowing an orally administered agent to be more readily absorbed into blood) or which enhance delivery of the parent agent to a biological compartment (e.g., the brain or lymphatic system). In various embodiments, pharmaceutically acceptable salts described herein include, by way of non-limiting example, an Nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

The term “recruiting of monocytes” as described herein includes the migration of monocytes into or out of the endothelium, their attachment and propagation, for example, into endothelial fissures. The attachment of monocytes is also known as monocyte adhesion, or as monocyte arrest when the attachment occurs in shear flow as under physiological conditions, for example, in blood capillaries, microvascular or arterial streamlines.

By the term “polypeptide” is meant synthetic or nonsynthetic peptide compounds, as well as purified, modified fragments of natural proteins, native forms or recombinant peptides or proteins. The term “polypeptide” likewise includes pharmacologically acceptable salts, pharmacologically acceptable derivatives and/or conjugates of the corresponding polypeptide.

Pharmacologically acceptable derivatives include, for example, esters, amides, N-acyl and/or O-acyl derivatives, carboxylated, acetylated, phosphorylated and/or glycosylated polypeptides. Conjugates include, for example, sugar or polyethylene glycol conjugates, biotinylated, radioactively or fluorescently labeled polypeptides.

The term “peptide mimetic”, “mimetic peptide” and “analog” are used herein interchangeably for the purposes of the specifications and claims, to mean a peptide that mimics part or all of the bioactivity of an endogenous protein ligand. In one embodiment, peptide mimetics are modeled after a specific peptide and display an altered peptide backbone, altered amino acids and/or an altered primary amino acid sequence when compared to the peptide of which is was designed to mimic.

As used herein, the terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and immunoglobulin are used interchangeably in the broadest sense. The subunit structures and three-dimensional configurations of the different classes of immunoglobulins are well known in the art. In some embodiments an antibody is part of a larger fusion molecule, formed by covalent or non-covalent association of the antibody with one or more other proteins or peptides.

As used herein, the term “derivative” in the context of a polypeptide or protein, e.g. an antibody, refers to a polypeptide or protein that comprises an amino acid sequence which has been altered by the introduction of amino acid residue substitutions, deletions or additions. The term “derivative” as used herein also refers to a polypeptide or protein which has been modified, i.e., by the covalent attachment of any type of molecule to the antibody. For example, in some embodiments a polypeptide or protein is modified, e.g., by glycosylation, acetylation, pegylation, phosphorylation, amidation, derivatization by known protecting/blocking groups, proteolytic cleavage, linkage to a cellular ligand or other protein, etc. In some embodiments, derivatives, polypeptides or proteins are produced by chemical modifications using techniques known to those of skill in the art, including, but not limited to specific chemical cleavage, acetylation, formylation, metabolic synthesis of tunicamycin, etc. In some embodiments a derivative a polypeptide or protein possesses a similar or identical function as the polypeptide or protein from which it was derived.

The terms “full length antibody”, “intact antibody” and “whole antibody” are used herein interchangeably, to refer to an antibody in its substantially intact form, and not antibody fragments as defined below. These terms particularly refer to an antibody with heavy chains contains Fc regions. In some embodiments an antibody variant of the invention is a full length antibody. In some embodiments the full length antibody is human, humanized, chimeric, and/or affinity matured.

An “affinity matured” antibody is one having one or more alteration in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by known procedures. See, for example, Marks et al., (1992) Biotechnology 10:779-783 that describes affinity maturation by variable heavy chain (VH) and variable light chain (VL) domain shuffling. Random mutagenesis of CDR and/or framework residues is described in: Barbas, et al. (1994) Proc. Nat. Acad. Sci, USA 91:3809-3813; Shier et al., (1995) Gene 169:147-155; Yelton et al., 1995, J. Immunol. 155:1994-2004; Jackson et al., 1995, J. Immunol. 154(7):3310-9; and Hawkins et al, (19920, J. Mol. Biol. 226:889-896, for example.

The terms “binding fragment”, “antibody fragment” or “antigen binding fragment” are used herein, for purposes of the specification and claims, to mean a portion or fragment of an intact antibody molecule, preferably wherein the fragment retains antigen-binding function. Examples of antibody fragments include Fab, Fab′, F(ab′)₂, Fd, Fd′ and Fv fragments, diabodies, linear antibodies (Zapata et al. (1995) Protein Eng. 10: 1057), single-chain antibody molecules, single-chain binding polypeptides, scFv, bivalent scFv, tetravalent scFv, and bispecific or multispecific antibodies formed from antibody fragments.

“Fab” fragments are typically produced by papain digestion of antibodies resulting in the production of two identical antigen-binding fragments, each with a single antigen-binding site and a residual “Fc” fragment. Pepsin treatment yields a F(ab′)2 fragment that has two antigen-combining sites capable of cross-linking antigen. An “Fv” is the minimum antibody fragment that contains a complete antigen recognition and binding site. In a two-chain Fv species, this region consists of a dimer of one heavy- and one light-chain variable domain in tight, non-covalent association. In a single-chain Fv (scFv) species, one heavy- and one light-chain variable domain are covalently linked by a flexible peptide linker such that the light and heavy chains associate in a “dimeric” structure analogous to that in a two-chain Fv species. It is in this configuration that the three CDRs of each variable domain interact to define an antigen-binding site on the surface of the VH-VL dimer Collectively, the six CDRs confer antigen-binding specificity to the antibody. However, even a single variable domain (or half of an Fv comprising only three CDRs specific for an antigen) has the ability to recognize and bind antigen, although usually at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known. Methods for producing the various fragments from monoclonal Abs are well known to those skilled in the art (see, e.g., Plü ckthun, 1992, Immunol Rev. 130:152-188).

The term “monoclonal antibody” as used herein refers to an antibody obtained from a population of substantially homogeneous antibodies, i.e., the individual antibodies comprising the population are identical except for possible naturally occurring mutations that are present in minor amounts. In some embodiments monoclonal antibodies are made, for example, by the hybridoma method first described by Kö hler and Milstein (1975) Nature 256:495, or are made by recombinant methods, e.g., as described in U.S. Pat. No. 4,816,567. In some embodiments monoclonal antibodies are isolated from phage antibody libraries using the techniques described in Clackson et al., Nature 352:624-628 (1991), as well as in Marks et al., J. Mol. Biol. 222:581-597 (1991).

As used herein, the term “epitope” refers to a fragment of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method, for example by immunoassays. Antigenic epitopes need not necessarily be immunogenic.

The phrase “specifically binds” when referring to the interaction between an antibody or other binding molecule and a protein or polypeptide or epitope, typically refers to an antibody or other binding molecule that recognizes and detectably binds with high affinity to the target of interest. Preferably, under designated or physiological conditions, the specified antibodies or binding molecules bind to a particular polypeptide, protein or epitope yet does not bind in a significant or undesirable amount to other molecules present in a sample. In other words the specified antibody or binding molecule does not undesirably cross-react with non-target antigens and/or epitopes. Further, in some embodiments, an antibody that specifically binds, binds through the variable domain or the constant domain of the antibody. For the antibody that specifically binds through its variable domain, it is not aggregated, i.e., is monomeric. A variety of immunoassay formats are used to select antibodies or other binding molecule that are immunoreactive with a particular polypeptide and have a desired specificity. For example, solid-phase ELISA immunoassays, BIAcore, flow cytometry and radioimmunoassays are routinely used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that are used to determine or assess immunoreactivity and specificity. “Selective binding”, “selectivity”, and the like refer the preference of a antibody to interact with one molecule as compared to another. Preferably, interactions between antibodies, particularly modulators, and proteins are both specific and selective. Note that in some embodiments a small antibody is designed to “specifically bind” and “selectively bind” two distinct, yet similar targets without binding to other undesirable targets.

As used herein, “transplant complications” means an immune-mediated complication resulting from an organ, a plurality of cells, and/or a tissue transplant. For example, a “transplant complication” can result from the recipient's immune system mounting an immune response against the transplanted organ, tissue, and/or plurality of cells. A “transplant complication” also includes an immune response mounted against the recipient by the immune cells from the transplanted organ, plurality of cells, and/or tissue. “Transplant complications” includes hyperacute transplant rejection, acute transplant rejection, chronic transplant rejection, acute graft-versus-host disease, chronic graft-versus-host disease, or combinations thereof.

### II. INFLAMMATORY DISORDERS

In some embodiments, the methods and compositions described herein treat inflammation (e.g., acute or chronic). In certain instances, inflammation results from (either partially or fully) an infection. In certain instances, inflammation results from (either partially or fully) damage to a tissue (e.g., by a burn, by frostbite, by exposure to a cytotoxic agent, or by trauma). In certain instances, inflammation results from (either partially or fully) an autoimmune disorder. In certain instances, inflammation results from (either partially or fully) the presence of a foreign body (e.g., a splinter). In certain instances, inflammation results from exposure to a toxin and/or chemical irritant.

As used herein, “acute inflammation” refers to inflammation characterized in that it develops over the course of a few minutes to a few hours, and ceases once the stimulus has been removed (e.g., an infectious agent has been killed by an immune response or administration of a therapeutic agent, a foreign body has been removed by an immune response or extraction, or damaged tissue has healed). The short duration of acute inflammation results from the short half-lives of most inflammatory mediators.

In certain instances, acute inflammation begins with the activation of leukocytes (e.g., dendritic cells, endothelial cells and mastocytes). In certain instances, the leukocytes release inflammatory mediators (e.g., histamines, proteoglycans, serine proteases, eicosanoids, and cytokines). In certain instances, inflammatory mediators result in (either partially or fully) the symptoms associated with inflammation. For example, in certain instances an inflammatory mediator dilates post capillary venules, and increases blood vessel permeability. In certain instances, the increased blood flow that follows vasodilation results in (either partially or fully) rubor and calor. In certain instances, increased permeability of the blood vessels results in an exudation of plasma into the tissue leading to edema. In certain instances, the latter allows leukocytes to migrate along a chemotactic gradient to the site of the inflammatory stimulant. Further, in certain instances, structural changes to blood vessels (e.g., capillaries and venules) occur. In certain instances, the structural changes are induced (either partially or fully) by monocytes and/or macrophages. In certain instances, the structural changes include, but are not limited to, remodeling of vessels, and angiogenesis. In certain instances, angiogenesis contributes to the maintenance of chronic inflammation by allowing for increased transport of leukocytes. Additionally, in certain instances, histamines and bradykinin irritate nerve endings leading to itching and/or pain.

In certain instances, chronic inflammation results from the presence of a persistent stimulant (e.g., persistent acute inflammation, bacterial infection (e.g., by Mycobacterium tuberculosis), prolonged exposure to chemical agents (e.g., silica, or tobacco smoke) and autoimmune reactions (e.g., rheumatoid arthritis)). In certain instances, the persistent stimulant results in continuous inflammation (e.g., due to the continuous recruitment of monocytes, and the proliferation of macrophages). In certain instances, the continuous inflammation further damages tissues which results in the additional recruitment of mononuclear cells thus maintaining and exacerbating the inflammation. In certain instances, physiological responses to inflammation further include angiogenesis and fibrosis.

Multiple disorders are associated with inflammation (i.e., inflammatory disorders). Inflammatory disorders include, but are not limited to, Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjë gren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, or combinations thereof.

In some embodiments, the methods and compositions described herein treat a T-cell mediated autoimmune disorder. In certain instances, a T-cell mediated autoimmune disorder is characterized by a T-cell mediated immune response against self (e.g., native cells and tissues).

Examples of T-cell mediated autoimmune disorders include, but are not limited to colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjë gren's syndrome, systemic lupus erythematosus (SLE), vasculitis syndromes (e.g., giant cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In some embodiments, the methods and compositions described herein treat pain. Pain includes, but is not limited to acute pain, acute inflammatory pain, chronic inflammatory pain and neuropathic pain.

In some embodiments, the methods and compositions described herein treat hypersensitivity. As used herein, “hypersensitivity” refers to an undesirable immune system response. Hypersensitivity is divided into four categories. Type I hypersensitivity includes allergies (e.g., Atopy, Anaphylaxis, or Asthma). Type II hypersensitivity is cytotoxic/antibody mediated (e.g., Autoimmune hemolytic anemia, Thrombocytopenia, Erythroblastosis fetalis, or Goodpasture's syndrome). Type III is immune complex diseases (e.g., Serum sickness, Arthus reaction, or SLE). Type IV is delayed-type hypersensitivity (DTH), Cell-mediated immune memory response, and antibody-independent (e.g., Contact dermatitis, Tuberculin skin test, or Chronic transplant rejection).

As used herein, “allergy” means a disorder characterized by excessive activation of mast cells and basophils by IgE. In certain instances, the excessive activation of mast cells and basophils by IgE results (either partially or fully) in an inflammatory response. In certain instances, the inflammatory response is local. In certain instances, the inflammatory response results in the narrowing of airways (i.e., bronchoconstriction). In certain instances, the inflammatory response results in inflammation of the nose (i.e., rhinitis). In certain instances, the inflammatory response is systemic (i.e., anaphylaxis).

In some embodiments, the methods and compositions described herein treat angiogenesis. As used herein, “angiogenesis” refers to the formations of new blood vessels. In certain instances, angiogenesis occurs with chronic inflammation. In certain instances, angiogenesis is induced by monocytes and/or macrophages.

In some embodiments the present invention comprises a method of treating a neoplasia. In certain instances, a neoplastic cell induces an inflammatory response. In certain instances, part of the inflammatory response to a neoplastic cell is angiogenesis. In certain instances, angiogenesis facilitates the development of a neoplasia. In some embodiments, the neoplasia is: angiosarcoma, Ewing sarcoma, osteosarcoma, and other sarcomas, breast carcinoma, cecum carcinoma, colon carcinoma, lung carcinoma, ovarian carcinoma, pharyngeal carcinoma, rectosigmoid carcinoma, pancreatic carcinoma, renal carcinoma, endometrial carcinoma, gastric carcinoma, liver carcinoma, head and neck carcinoma, breast carcinoma and other carcinomas, Hodgkins lymphoma and other lymphomas, malignant and other melanomas, parotid tumor, chronic lymphocytic leukemia and other leukemias, astrocytomas, gliomas, hemangiomas, retinoblastoma, neuroblastoma, acoustic neuroma, neurofibroma, trachoma and pyogenic granulomas.

In some embodiments, the methods and compositions described herein treat obesity. As used herein, “obesity” means an accumulation of adipose tissue with a BMI of greater than or equal to 30 kg/m². In certain instances, obesity is characterized a proinflammatory state, increasing the risk of thrombosis. In certain instances, obesity is associated with a low-grade inflammation of white adipose tissue (WAT). In certain instances, WAT associated with obesity is characterized by an increased production and secretion of a wide range of inflammatory molecules including TNF-alpha and interleukin-6 (IL-6). In certain instances, WAT is infiltrated by macrophages, which produce pro-inflammatory cytokines. In certain instances, TNF-alpha is overproduced in adipose tissue. In certain instances, IL-6 production increases during obesity.

In some embodiments, the methods and compositions described herein treat metabolic syndrome. In certain instances, metabolic syndrome is associated with fasting hyperglycemia; high blood pressure; central obesity; decreased HDL levels; elevated triglyceride levels; systemic inflammation; or combinations thereof. In certain instances, metabolic syndrome is characterized by an increase in the levels of C-reactive protein, fibrinogen, (IL-6), and TNFα.

### AAA

In certain instances, an atherosclerotic plaque results (partially or fully) in the development of an aneurysm. In some embodiments, the methods and compositions described herein treat an aneurysm. In some embodiments, the methods and compositions described herein treat an abdominal aortic aneurysm (“AAA”). As used herein, an “abdominal aortic aneurysm” is a localized dilatation of the abdominal aorta. In certain instances, the rupture of an AAA results in bleeding, leading to hypovolemic shock with hypotension, tachycardia, cyanosis, and altered mental status.

In some embodiments, the compositions and methods disclosed herein treat abdominal aortic aneurysms. In certain instances, abdominal aortic aneurysms result (partially or fully) from an extensive breakdown of structural proteins (e.g., elastin and collagen). In some embodiments, a method and/or composition disclosed herein partially or fully inhibits the breakdown of a structural protein (e.g., elastin and collagen). In certain instances, the breakdown of structural proteins is caused by activated MMPs. In some embodiments, a method and/or composition disclosed herein partially or fully inhibits the activation of an MMP. In some embodiments, a composition and/or method disclosed herein inhibit the upregulation of MMP-1, MMP-9 or MMP-12. In certain instances, MIF is co-expressed with MMP-1, MMP-9, and MMP-12 in abdominal aortic aneurysms. In certain instances, the MIF is upregulated in stable abdominal aortic aneurysm and is intensified further in ruptured aneurysms. In certain instances, MMPs are activated following infiltration of a section of the abdominal aorta by leukocytes (e.g., macrophages and neutrophils). In some embodiments, a method and/or composition disclosed herein partially or fully inhibits the activity of MIF. In some embodiments, a method and/or composition disclosed herein partially or fully inhibits the infiltration of a section of the abdominal aorta by leukocytes.

### Anti-Inflammatory Agents

The terms “anti-inflammatory agent” and “modulator of inflammation” are used interchangeably. As used herein, the terms refer to agents treat inflammation and/or an inflammatory disorder. In some embodiments, the anti-inflammatory agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent (e.g., cyclosporine A), an alkylating agent, an anti-metabolite, a plant alkaloid, a terpenoids, a topoisomerase inhibitor, an antitumour antibiotic, an antibody, a hormonal therapy (e.g., aromatase inhibitors), a leukotriene inhibitor, or combinations thereof.

In some embodiments, the second anti-inflammatory agent is: cyclosporine A, alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), ), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Actos® (Pioglitazone), Avandia® (Rosiglitazone), Amaryl® (Glimepiride), Sulfonylurea-types, Diabeta® (Glyburide), Diabinese® (Chlorpropamide), Glucotrol® (Glipizide), Glynasec (glyburide), Micronase® (glyburide), Orinase® (Tolbutamide), Tolinase® (Tolazamide), Glucophage, Riomet® (Metformin), Glucovance® (glyburide+metformin), Avandamet® (Rosiglitazone+metformin), Avandaryl® (Rosiglitazone+glimepiride), Byetta® (Exenatide), Insulins, Januvia® (Sitagliptin), Metaglip® (glipizide and metformin), Prandin® (Repaglinide), Precose® (Acarbose), Starlix® (Nateglinide), Xenical® (Orlistat), cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S—[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-C5 antibody (e.g., eculizumab); doxycycline; or combinations thereof.

### III. MODULATORS OF LIPIDS

The term “modulator of a lipid” as used herein refers to an agent that modulates the concentration of lipid in an individual. In some embodiments, the modulator of a lipid modulates HDL and/or LDL concentrations. In some embodiments, the methods and compositions described herein treat inflammation associated with administration of an agent that modulates a lipid.

In certain instances, ApoA-I is overexpressed in RA patients that respond to anti-inflammatory Infliximab treatment. In certain instances, infliximab modulates HDL levels in RA patients. In certain instances, (HDL)-associated A-I is also a specific inhibitor of cytokine production in monocytes & macrophages upon contact with stimulated T cells. In certain instances, ApoA-1-HDL is a negative acute-phase protein (lowered by more than 25% during the acute phase). In certain instances, A-1-HDL act as constitutive anti-inflammatory factor.

### Lipids and Lipoproteins

### HDL

HDL is a type of lipoprotein that transports cholesterol and triglycerides to the liver. In certain instances, HDL comprises ApoA1 and ApoA2. In certain instances, ApoA1 and ApoA2 are expressed in the liver. In certain instances, the liver synthesized HDL.

In certain instances, HDL transport cholesterol from cells to the liver, adrenals, ovary and/or testes. In certain instances, cholesterol transported to the liver is excreted as bile. In certain instances, cholesterol transported to adrenals, ovaries and/or testes are used to synthesize steroid hormones.

HDL comprises multiple sub-classes of lipoprotein. In certain instances, the subclasses of HDL differ in size, density, protein and lipid composition. In certain instances, some HDL are protective, anti-oxidative, anti-inflammatory and/or anti-atherogenic. In certain instances, some HDL are neutral. In certain instances, some HDL enhance oxidation, increase inflammation and/or are pro-atherogenic.

In certain instances, increasing the concentration of HDL across all or most sub-classes results in the production of reactive oxygen species (ROS). In certain instances, an enzyme associated with HDL modifies a phospholipid into an oxidized phospholipid. In certain instances, an enzyme associated with HDL modifies a cholesterol into an oxidized sterol. In certain instances, an oxidized sterol and/or an oxidized phospholipid results in pro-inflammatory and/or pro-atherogenic HDL.

In certain instances, cholesteryl ester transfer protein (CETP) exchanges triglycerides transported by VLDL (very low density lipoprotein) for cholesteryl esters transported by HDL. In certain instances, the exchange of triglycerides for cholesteryl esters results in VLDL being processed into LDL. In certain instances, LDL is removed from circulation by the LDL receptor pathway. In certain instances, the triglycerides are degraded by hepatic lipase. In certain instances, delipidified HDL recirculate in the blood and transport additional lipids to the liver.

In certain instances, inhibiting CETP disrupts the metabolism of HDL. In certain instances, inhibiting CETP prevents transfer of HDL-cholesterol and increases circulating levels of cholesteryl-ester enriched (larger) HDL subtractions. In some embodiments, inhibiting (partially or fully) CETP treat CVD. In certain instances, slowing the catabolism of HDL increases total circulating HDL levels. In certain instances, increasing total circulating HDL levels treats atherogenesis. In some embodiments, inhibiting (partially or fully) CETP results (partially or fully) in inflammation and/or worsening of CVD. In certain instances, increasing total circulating HDL levels generates a lipid pool with reduced clearance (kinetics). In certain instances, reduced clearance of lipids increases HDL capacity to harbor oxidizable and potentially inflammatory lipid stores.

### LDL

Low-density lipoprotein (LDL) is a type of lipoprotein that transports cholesterol and triglycerides from the liver to peripheral tissues. In certain instances, LDL comprises an apolipoprotein B (ApoB). In certain instances, ApoB is expressed as two isoforms, ApoB48 and ApoB100. In certain instances, ApoB48 is synthesized by intestinal cells. In certain instances, ApoB100 is synthesized in the liver. In certain instances, Hsp110 stabilizes of ApoB.

### Lipid Modulating Agents

In some embodiments, the agent used to modulate a lipid and/or a lipoprotein include fibrates; apolipoprotein A-1 modulators; CETP modulators; or combinations thereof.

In some embodiments, the agent used to modulate a lipid and/or a lipoprotein increases the concentration of HDL. In some embodiments, the cardiovascular disorder agent is bezafibrate; ciprofibrate; clofibrate; gemfibrozil; fenofibrate; or combinations thereof.

In some embodiments, the agent used to modulate a lipid and/or a lipoprotein selectively increases the levels of apoA1 protein (e.g. by transcriptional induction of the gene encoding apoA1) and increases the production of nascent HDL (apoA1-enriched). In some embodiments, the second active agent is DF4 (Ac-D-W-F-K-A-F-Y-D-K-V-A-E-K-F-K-E-A-F—NH2); DF5; RVX-208 (Resverlogix); or combinations thereof.

In some embodiments, the agent used to modulate a lipid and/or a lipoprotein inhibits (partially or completely) the activity of Cholesteryl Ester Transfer Protein (CETP). In some embodiments, the cardiovascular disorder agent increases HDL-C concentration In some embodiments, the cardiovascular disorder agent increases antioxidant enzymes associated with HDL and decreases oxidized LDL. In some embodiments, the cardiovascular disorder agent is torcetrapib; anacetrapid; JTT-705 (Japan Tobacco/Roche); or combinations thereof.

### IV. MACROPHAGE MIGRATION INHIBITORY FACTOR (MIF)

In some embodiments, the methods and compositions disclosed herein inhibit (partially or fully) the activity of MIF. MIF is a pro-inflammatory lymphokine. In certain instances, it is secreted by a lymphocyte (e.g. a T-cell) in response to an infection, inflammation, or tissue injury. In certain instances, MIF is a functional noncognate ligand for the receptors CXCR2 and CXCR4. In some embodiments, the methods and compositions disclosed herein inhibit (partially or fully) the activity of CXCR2 and/or CXCR4.

In certain instances, MIF induces chemotaxis in nearby leukocytes (e.g. lymphocytes, granulocytes and monocytes/macrophages) along a MIF gradient. In certain instances, MIF induces the chemotaxis of a leukocyte (e.g. lymphocytes, granulocytes and monocytes/macrophages) to the site of an infection, inflammation or tissue injury. In certain instances, the chemotaxis of a leukocyte (e.g. lymphocytes, granulocytes and monocytes/macrophages) along a MIF gradient results in inflammation at the site of infection, inflammation, or tissue injury.

In certain instances, a human MIF polypeptide is encoded by a nucleotide sequence located on chromosome 22 at the cytogenic band 22q11.23. In certain instances, a MIF protein is a 12.3 kDa protein. In certain instances, a MIF protein is a homotrimer comprising three polypeptides of 115 amino acids. In certain instances, a MIF protein comprises a pseudo-ELR motif that mimics the ELR motif found in chemokines. In certain instances, a pseudo-ELR motif of a MIF protein mediates binding to a CXCR2 and/or CXCR4 receptor. In certain instances, a MIF protein comprises a 10- to 20-residue N-terminal Loop motif (N-loop). In certain instances, a MIF N-loop mediates binding to a CXCR2 and/or CXCR4 receptor.

In some embodiments, the methods described herein comprise a CXCR2 antagonist; an anti-CXCR2 antibody; a CXCR4 antagonist; an anti-CXCR4 antibody; a MIF antagonist (e.g., a peptide, polypeptide, or small molecule); an anti-MIF antibody; or combinations thereof. In some embodiments, the antagonist inhibits the binding of MIF to CXCR2 and/or CXCR4 by binding to a pseudo-ELR motif of MIF. In some embodiments, the antagonist inhibits the binding of MIF to CXCR2 and/or CXCR4 by binding to an N-loop motif of MIF.

### A. Disruption of MIF Domains

In some embodiments, the modulator of MIF disrupts the ability of MIF to interact with CXCR2, CXCR4, CD74, or a combination thereof. In some embodiments, the ability of MIF to interact with CXCR2, CXCR4, CD74, or a combination thereof is inhibited by occupying, masking, or otherwise disrupting domains on MIF to which CXCR2, CXCR4, and/or CD74 bind (e.g., the N-loop and/or the pseudo-ELR loop).

In some embodiments, the ability of MIF to interact with CXCR2, CXCR4, CD74, or a combination thereof is inhibited by a small molecule, peptide, antibody, and/or peptibody occupying, masking, or otherwise disrupting domains on MIF to which CXCR2, CXCR4, and/or CD74 bind. In some embodiments, a small molecule, peptide, antibody, and/or peptibody inhibits MIF binding to CXCR2, CXCR4, and/or CD74. In certain instances, occupying, masking, or otherwise disrupting domains on MIF does not affect CXCR2 and CXCR4 signaling mediated by other agonists/ligands (e.g., IL-8/CXCL8, GRObeta/CXCL2 and/or Stromal Cell-Derived Factor-1a (SDF-1a)/CXCL12).

In certain instances, the pseudo-ELR region of MIF mediates ligand (e.g., CD74, CXCR2, CXCR4) binding to MIF. In some embodiments, the binding of a small molecule, peptide, antibody, and/or peptibody to a pseudo-ELR loop of MIF inhibits the ability of MIF to form a signaling complex with CXCR2, CXCR4, CD74, or a combination thereof. In some embodiments, the binding of a small molecule, peptide, antibody, and/or peptibody to a pseudo-ELR loop of MIF invokes a conformational change in MIF that prevents receptor or substrate interactions.

In certain instances, the N-loop region of MIF mediates ligand (e.g., CD74, CXCR2, CXCR4) binding to MIF. In some embodiments, the binding of a small molecule, peptide, antibody, and/or peptibody to an N-loop motif of MIF inhibits the ability of MIF to form a signaling complex with CXCR2, CXCR4, CD74, or a combination thereof. In some embodiments, the binding of a small molecule, peptide, antibody, and/or peptibody to an N-loop motif of MIF invokes a conformational change in MIF that prevents receptor or substrate interactions.

In certain instances, amino acids 65-94 of MIF (e.g., IGKIGGAQNRSYSKLLCGLLAERLRISPDR; numbering includes the first methionine) mediate CXCR2 binding to MIF. In some embodiments, the binding of a small molecule, peptide, antibody, and/or peptibody to amino acids 65-94 of MIF inhibits the ability of MIF to form a signaling complex with CXCR2. In some embodiments, the binding of a peptide to amino acids 65-94 of MIF inhibits the ability of MIF to form a signaling complex with CXCR2. In some embodiments, the binding of an antibody to amino acids 65-94 of MIF inhibits the ability of MIF to form a signaling complex with CXCR2. In some embodiments, the binding of a peptibody to amino acids 65-94 of MIF inhibits the ability of MIF to form a signaling complex with CXCR2. In some embodiments, the binding of a small molecule to amino acids 65-94 of MIF inhibits the ability of MIF to form a signaling complex with CXCR2.

In certain instances, amino acids 80-95 of MIF (e.g., LCGLLAERLRISPDRV; numbering includes the first methionine) mediate ligand binding to MIF. In some embodiments, the binding of a small molecule, peptide, antibody, and/or peptibody to amino acids 80-95 of MIF inhibits the ability of MIF to form a signaling complex with a ligand. In some embodiments, the binding of a peptide to amino acids 80-95 of MIF inhibits the ability of MIF to form a signaling complex with a ligand. In some embodiments, the binding of an antibody to amino acids 80-95 of MIF inhibits the ability of MIF to form a signaling complex with a ligand. In some embodiments, the binding of a peptibody to amino acids 80-95 of MIF inhibits the ability of MIF to form a signaling complex with a ligand. In some embodiments, the binding of a small molecule to amino acids 80-95 of MIF inhibits the ability of MIF to form a signaling complex with a ligand.

In some embodiments, the modulator of MIF is a peptide that occupies, masks, or otherwise disrupts a domain on MIF to which CXCR2, CXCR4, and/or CD74 binds. In some embodiments, the peptide specifically binds to all or a portion of the pseudo-ELR loop of MIF. In some embodiments, the peptide specifically binds to all or a portion of the N-loop motif of MIF. In some embodiments, the peptide specifically binds to all or a portion of both the pseudo-ELR and N-loop motifs.

In some embodiments, the modulator of MIF is a peptide that specifically binds to all or a portion of a peptide sequence as follows: VNTNVPPRASVPDGFLSELTQQLAQATGKPPQYIAVHVVPDQL and the corresponding feature/domain of at least one of a MIF monomer or MIF trimer; a peptide that specifically binds to all or a portion of a peptide sequence as follows: PDQLMAFGGSSEPCALCSL and the corresponding feature/domain of at least one of a MIF monomer or MIF trimer; a peptide that specifically binds to all or a portion of a peptide sequence as follows: VNTNVPPRASVPDGFLSELTQQLAQATGKPPQYIAVHVVPDQLMAFGGSSEPCALCSL and the corresponding feature/domain of at least one of a MIF monomer or MIF trimer; a peptide that specifically binds to all or a portion of a peptide sequence as follows: PDQLMAFGGSSEPCALCSLHSI and the corresponding feature/domain of at least one of a MIF monomer or MIF trimer; or combinations thereof.

In some embodiments, the modulator of MIF is an antibody that occupies, masks, or otherwise disrupts a domain on MIF to which CXCR2, CXCR4, and/or CD74 binds. In some embodiments, the antibody specifically binds to all or a portion of the pseudo-ELR loop of MIF. In some embodiments, the antibody specifically binds to all or a portion of the N-loop motif of MIF. In some embodiments, the antibody specifically binds to all or a portion of both the pseudo-ELR and N-loop motifs.

In some embodiments, the modulator of MIF is a peptibody that occupies, masks, or otherwise disrupts a domain on MIF to which CXCR2, CXCR4, and/or CD74 binds. In some embodiments, the peptibody specifically binds to all or a portion of the pseudo-ELR loop of MIF. In some embodiments, the peptibody specifically binds to all or a portion of the N-loop motif of MIF. In some embodiments, the peptibody specifically binds to all or a portion of both the pseudo-ELR and N-loop motifs.

In some embodiments, the modulator of MIF is a small molecule that occupies, masks, or otherwise disrupts a domain on MIF to which CXCR2, CXCR4, and/or CD74 binds. In some embodiments, the small molecule specifically binds to all or a portion of the pseudo-ELR loop of MIF. In some embodiments, the small molecule specifically binds to all or a portion of the N-loop motif of MIF. In some embodiments, the small molecule specifically binds to all or a portion of both the pseudo-ELR and N-loop motifs.

### B. Disruption of CXCR2 and CXCR4 Domains

In some embodiments, the modulator of MIF is an agent that occupies, masks, or otherwise disrupts a domain on CXCR2 and/or CXCR4 to which MIF and/or CD74 bind. In some embodiments, the modulator of MIF is an agent that disrupts the ability of MIF to form a signaling complex with CXCR2, CXCR4, CD74, or a combination thereof.

In some embodiments, the agent that inhibits the binding of MIF and/or CD74 to CXCR2 and/or CXCR4 is a peptide.

In some embodiments, the agent that inhibits the binding of MIF and/or CD74 to CXCR2 and/or CXCR4 is an antibody.

In some embodiments, the agent that inhibits the binding of MIF and/or CD74 to CXCR2 and/or CXCR4 is a peptibody.

In some embodiments, the agent that inhibits the binding of MIF and/or CD74 to CXCR2 and/or CXCR4 is a derivative of hydroxycinnamate, Schiff-based tryptophan analogs, or imino-quinone metabolites of acetaminophen.

In some embodiments, the agent that inhibits the binding of MIF and/or CD74 to CXCR and/or CXCR4 is glyburide, probenicide, DIDS (4,4-diisothiocyanatostilbene-2,2-disulfonic acid), bumetanide, furosemide, sulfobromophthalein, diphenylamine-2-carboxylic acid, flufenamic acid, or combinations thereof.

In some embodiments, the agent that inhibits the binding of MIF and/or CD74 to CXCR2 is CXCL8₍₃₋₇₄₎K11R/G31P; IL-8₍₄₋₇₂₎; IL-8₍₆₋₇₂₎; recombinant IL-8 (rIL-8); recombinant IL-8, NMeLeu (rhIL-8 with an N-methylated leucine at position 25); (AAR)IL-8 (IL-8 with N-terminal Ala-4-Ala5 instead of Glu-4-Leu5); GRO-alpha₍₁₋₇₃₎ (also known as CXCL1); GRO-alpha₍₄₋₇₃₎; GRO-alpha₍₅₋₇₃₎; GRO-alpha₍₆₋₇₃₎; recombinant GRO (rGRO); (ELR)PF4 (PF4 with an ELR seq. at the N-terminus); recombinant PF4 (rPF4); Antileukinate; Sch527123 (-hydroxy-N,N-dimethyl-3-{2-[[(R)-1-(5-methyl-furan-2-yl)-propyl]amino]-3,4-dioxo-cyclobut-1-enylamino}-benzamide); N-(3-(aminosulfonyl)-4-chloro-2-hydroxyphenyl)-N′-(2,3-dichlorophenyl) urea; SB-517785-M (GSK); SB 265610 (N-(2-Bromophenyl)-N′-(7-cyano-1H-benzotriazol-4-yl)urea); SB225002 (N-(2-Bromophenyl)-N′-(2-hydroxy-4-nitrophenyl) urea); SB455821 (GSK), SB272844 (GSK); DF2162 (4-[(1R)-2-amino-1-methyl-2-oxoethyl]phenyl trifluoromethanesulphonate); Reparixin; or combinations thereof.

In some embodiments, the agent that inhibits the binding of MIF and/or CD74 to CXCR4 is ALX40-4C(N-alpha-acetyl-nona-D-arginine amide acetate); AMD-070 (AMD11070, AnorMED); Plerixafor (AMD3100); AMD3465(AnorMED); AMD8664 (1-pyridin-2-yl-N-[4-(1,4,7-triazacyclotetradecan-4-ylmethyl)benzyl]methanamine); KRH-1636 (Kureha Chemical Industry Co. Limited); KRH-2731 (Kureha Chemical Industry Co. Limited); KRH-3955 (Kureha Chemical Industry Co. Limited); KRH-3140 (Kureha Chemical Industry Co. Limited); T134 (L-citrulline 16-TW70 substituted for the C-terminal amide by a carboxylic acid); T22 ([Tyr^{5,12}, Lys⁷]-polyphemusin II); TW70 (des-[Cys8,13, Tyr9,12]-[D-Lys10, Pro11]-T22); T140 (H-Arg-Arg-Nal-Cys-Tyr-Arg-Lys-DLys-Pro-Tyr-Arg-Cit-Cys-Arg-OH); TC14012 (R—R-Nal-C—Y-(L)Cit-K-(D)Cit-P—Y-R-(L)citrulline-C—R—NH2, where Nal=L-3-(2-naphthylalanine), Cit=citruline and the peptide is cyclized with the cysteines); TN14003; RCP168 (vMIP-II₍₁₁₋₇₁₎ with D-amino acids added to the N terminus); POL3026 (Arg(*)-Arg-Nal(2)-Cys(1×)-Tyr-Gln-Lys-(d-Pro)-Pro-Tyr-Arg-Cit-Cys(1×)-Arg-Gly-(d-Pro)(*)); POL2438; compound 3 (N-(1-methyl-1-phenylethyl)-N-[((3S)-1-{2-[5-(4H-1,2,4-triazol-4-yl)-1H-indol-3-yl]ethyl}pyrrolidin-3-yl)methyl]amine); isothioureas 1a-1u (for information regarding isothioureas 1a-1u see Gebhard Thoma, et al., Orally Bioavailable Isothioureas Block Function of the Chemokine Receptor CXCR4 In Vitro and In Vivo, J. Med. Chem., Article ASAP (2008), which is herein incorporated by reference for such disclosures); or combinations thereof.

In some embodiments, the agent that inhibits the binding of MIF and/or CD74 to CXCR2 and/or CXCR4 is MIF is COR100140 (Genzyme Corp/Cortical Pty Ltd.); ISO-1 ((S,R)-3-(4-Hydroxyphenyl)-4,5-dihydro-5-isoxazole acetic acid, methyl ester); 4-IPP (4-iodo-6-phenylpyrimidine); or combinations thereof.

### C. Disruption of CD74 Domains

In some embodiments, the modulator of MIF is an agent that occupies, masks, or otherwise disrupts a domain on CD74 to which MIF, CXCR2, and/or CXCR4 bind. In some embodiments, the modulator of MIF is an agent that disrupts the ability of MIF to form a signaling complex with CXCR2, CXCR4, CD74, or a combination thereof.

In some embodiments, the agent that inhibits the binding of MIF, CXCR2, CXCR4, or a combination thereof to CD74 is a peptide.

In some embodiments, the agent that inhibits the binding of MIF, CXCR2, CXCR4, or a combination thereof to CD74 is an antibody. In some embodiments, the agent that inhibits the binding of MIF, CXCR2, CXCR4, or a combination thereof to CD74 is M-B741, 555538 (BD Pharmingen).

In some embodiments, the agent that inhibits the binding of MIF, CXCR2, CXCR4, or a combination thereof to CD74 is a peptibody.

In some embodiments, the agent that inhibits the binding of MIF, CXCR2, CXCR4, or a combination thereof to CD74 is a small molecule.

In certain instances, occupying, masking, or otherwise disrupting domains on MIF does not affect CD74 signaling mediated by other agonists/ligands (e.g., IL-8/CXCL8, GRObeta/CXCL2 and/or Stromal Cell-Derived Factor-1a (SDF-1a)/CXCL12).

### D. MIF Mimics

In some embodiments, the modulator of MIF is an agent that disrupts the ability of MIF to form a signaling complex with CXCR2, CXCR4, CD74, or a combination thereof. In some embodiments, the modulator of MIF is a MIF-like peptide that mimics part or all of a MIF domain (e.g., the pseudo-ELR, or N-Loop domains). In some embodiments, the MIF-mimic binds to CXCR2, CXCR4, CD74, or a combination thereof and thus prevents CXCR2, CXCR4, or CD74 from binding to MIF.

In some embodiments, the MIF-mimic adopts structural or functional features similar to the N-Loop motif of MIF. In some embodiments, the MIF-mimic is a peptide. In some embodiments, the MIF-mimic comprises a peptide of Formula (I):

X¹-X²-Q/A-X³-X⁴-X⁵-X⁶-G/S-X⁷-X⁸-X⁹-X¹⁰-P-X¹¹

wherein:
X¹ is selected from the group consisting of threonine, glycine, proline and alanine;
X² is selected from the group consisting of glycine, asparagine, aspartic acid, and serine;
X³ is selected from the group consisting of methionine, isoleucine, leucine, alanine, proline, lysine, glutamine, arginine and lysine;
X⁴ is selected from the group consisting of methionine, isoleucine and leucine;
X⁵ is selected from the group consisting of alanine, threonine, methionine, serine and valine;
X⁶ is selected from the group consisting of phenylalanine, histidine, arginine and lysine;
X⁷ is selected from the group consisting of aspartic acid, glutamic acid, threonine, glycine and alanine;
X⁸ is selected from the group consisting of serine, threonine, lysine and arginine;
X⁹ is selected from the group consisting of serine, asparagine, glycine, threonine, aspartic acid, glutamic acid, glutamine and histidine;
X¹⁰ is selected from the group consisting of aspartic acid, glutamic acid, alanine and asparagine; and
X¹¹ is selected from the group consisting of cysteine, alanine, serine, threonine and valine.

In some embodiments, X¹ is proline. In some embodiments, X² is aspartic acid. In some embodiments, X³ is leucine. In some embodiments, X⁴ is methionine. In some embodiments, X⁵ is alanine. In some embodiments, X⁶ is phenylalanine. In some embodiments, X⁷ is glycine. In some embodiments, X⁸ is serine. In some embodiments, X⁹ is serine. In some embodiments, X¹⁰ is glutamic acid. In some embodiments, X¹¹ is serine cysteine.

In some embodiments, the MIF-mimic comprises any 5 or more consecutive peptide of Formula (I).

In some embodiments, the MIF-mimic comprises 5 or more consecutive amino acids of human MIF₄₄₋₅₇ (numbering includes the first methionine). In some embodiments, the MIF-mimic comprises 5 or more consecutive amino acids of murine MIF₄₄₋₅₇. In some embodiments, the MIF-mimic comprises 5 or more consecutive amino acids of porcine MIF₄₄₋₅₇. In some embodiments, the MIF-mimic comprises 5 or more consecutive amino acids of bovine MIF₄₄₋₅₇. In some embodiments, the MIF-mimic comprises 5 or more consecutive amino acids of rat MIF₄₄₋₅₇.

In some embodiments, the MIF-mimic comprises one or more of the peptides selected from Table 1. In some embodiments, the MIF-mimic comprises N- and/or C-terminal chemical modifications to improve ADME-PK. In some embodiments, MIF-mimic comprises non-natural amino acids. In some embodiments, MIF-mimic comprises cyclical variants.

TABLE 1
LMAFGGSSEPCALC
LMAFGGSSEPCAL
LMAFGGSSEPCA
LMAFGGSSEPC
LMAFGGSSEP
LMAFGGSSE
LMAFGGSS
LMAFGGS
LMAFGG
MAFGGSSEPCALC
MAFGGSSEPCAL
MAFGGSSEPCA
MAFGGSSEPC
MAFGGSSEP
MAFGGSSE
MAFGGSS
MAFGGS
AFGGSSEPCALC
AFGGSSEPCAL
AFGGSSEPCA
AFGGSSEPC
AFGGSSEP
AFGGSSE
AFGGSS
FGGSSEPCALC
FGGSSEPCAL
FGGSSEPCA
FGGSSEPC
FGGSSEP
FGGSSE
GGSSEPCALC
GGSSEPCAL
GGSSEPCA
GGSSEPC
GGSSEP
GSSEPCALC
GSSEPCAL
GSSEPCA
GSSEPC
SSEPCALC
GSSEPCALC
GSSEPCAL
GSSEPCA
GSSEPC
SSEPCALC
SSEPCAL
SSEPCA
SEPCALC
SEPCAL
EPCALC
QLMAFGGSSEPCALC
QLMAFGGSSEPCAL
QLMAFGGSSEPCA
QLMAFGGSSEPC
QLMAFGGSSEP
QLMAFGGSSE
QLMAFGGSS
QLMAFGGS
QLMAFGG
QLMAFG
CSSEPCALC (1096)
CFGGSSEPCALC (1081)
CLMAFGGSSEPCALC (1057)
CAFGGSSC (1079)
CLMAFGGSSEPC C(1059)
CAFGGSSEPCAC(1075)
CMAFGGSSEPC
CGGSSEPCAC
NVPRASVPD
VPDGFLSEL
CFGGSSEPC
IAVHVVPDQLMAFGGSSEPC
CLHSIGKIGGAQNRSYSKLL
PCALLCSLHSIGKIG
CSLHSIGKIGGAQNR
IGKIGGAQNRSYSKL
GAQNRSYSKLLCGLLA
CGLLAERLRISPDRV
ERLRISPDRVYINYY
cyclo(LMAFGGSSEPCALC)
cyclo(LMAFGGSSEPCAL)
cyclo(LMAFGGSSEPCA)
cyclo(LMAFGGSSEPC)
cyclo(LMAFGGSSEP)
cyclo(LMAFGGSSE)
cyclo(LMAFGGSS)
cyclo(LMAFGGS)
cyclo(LMAFGG)
cyclo(MAFGGSSEPCALC)
cyclo(MAFGGSSEPCAL)
cyclo(MAFGGSSEPCA)
cyclo(MAFGGSSEPC)
cyclo(MAFGGSSEP)
cyclo(MAFGGSSE)
cyclo(MAFGGSS)
cyclo(MAFGGS)
cyclo(GSSEPCALC)
cyclo(GSSEPCAL)
cyclo(GSSEPCA)
cyclo(GSSEPC)
cyclo(SSEPCALC)
cyclo(SSEPCAL)
cyclo(SSEPCA)
cyclo(SEPCALC)
cyclo(SEPCAL)
cyclo(EPCALC)
cyclo(QLMAFGGSSEPCALC)
cyclo(QLMAFGGSSEPCAL)
cyclo(QLMAFGGSSEPCA)
cyclo(QLMAFGGSSEPC)
cyclo(QLMAFGGSSEP)
cyclo(QLMAFGGSSE)
cyclo(QLMAFGGSS)
cyclo(QLMAFGGS)
cyclo(QLMAFGG)
cyclo(QLMAFG)
cyclo(AFGGSSEPCALC)
cyclo(AFGGSSEPCAL)
cyclo(AFGGSSEPCA)
cyclo(AFGGSSEPC)
cyclo(AFGGSSEP)
cyclo(AFGGSSE)
cyclo(AFGGSS)
cyclo(FGGSSEPCALC)
cyclo(FGGSSEPCAL)
cyclo(FGGSSEPCA)
cyclo(FGGSSEPC)
cyclo(FGGSSEP)
cyclo(FGGSSE)
cyclo(GGSSEPCALC)
cyclo(GGSSEPCAL)
cyclo(GGSSEPCA)
cyclo(GGSSEPC)
cyclo(GGSSEP)
cyclo(CSSEPCALC)
cyclo(CFGGSSEPCALC)
cyclo(CFGGSSEPCC)
cyclo(CFGGSSEPC)
cyclo(CGSSEPCALC)
cyclo(CAFGGSSEPCAC)
cyclo(CLMAFGGSSEPCALC)
cyclo(CAFGGSSC)

In some embodiments, the MIF-mimic adopts structural or functional features similar to the pseudo-ELR loop of MIF. In some embodiments, the MIF-mimic is a peptide.

In some embodiments, the MIF-mimic comprises a peptide of Formula (II):

X¹-X²-T/S—N-X³-X⁴-X⁵-X⁶-X⁷-X⁸-P/S-X⁹-X¹⁰

wherein:
X¹ is selected from the group consisting of valine, isoleucine, threonine, phenylalanine and leucine;
X² is selected from the group asparagine, arginine, aspartic acid, glutamic acid, serine and alanine;
X³ is selected from the group valine, isoleucine, arginine, lysine and leucine;
X⁴ is selected from the group proline, alanine, cysteine and leucine;
X⁵ is selected from the group arginine, lysine, glutamine, serine, alanine, aspartic acid, glutamic acid and asparagine;
X⁶ is selected from the group alanine, aspartic acid, glutamic acid, asparagine, serine and glutamine;
X⁷ is selected from the group serine, glutamic acid, aspartic acid, asparagine, arginine, glycine, lysine and arginine;
X⁸ is selected from the group valine, isoleucine and phenylalanine;
X⁹ is selected from the group aspartic acid, glutamic acid, valine, serine and threonine; and
X¹⁰ is selected from the group glycine, alanine, threonine, aspartic acid and glutamic acid.

In some embodiments, X¹ is valine. In some embodiments, X² is asparagine. In some embodiments, X³ is valine. In some embodiments, X⁴ is proline. In some embodiments, X⁵ is arginine. In some embodiments, X⁶ is alanine. In some embodiments, X⁷ is serine. In some embodiments, X⁸ is valine. In some embodiments, X⁹ is aspartic acid. In some embodiments, X¹⁰ is glycine.

In some embodiments, the MIF-mimic comprises any 5 or more consecutive peptide of Formula (II).

In some embodiments, the MIF-mimic comprises 5 or more consecutive amino acids of human MIF₁₋₄₅ (numbering includes the first methionine). In some embodiments, the MIF-mimic comprises 5 or more consecutive amino acids of murine MIF₁₋₄₅. In some embodiments, the MIF-mimic comprises 5 or more consecutive amino acids of porcine MIF₁₋₄₅. In some embodiments, the MIF-mimic comprises 5 or more consecutive amino acids of bovine MIF₁₋₄₅. In some embodiments, the MIF-mimic comprises 5 or more consecutive amino acids of rat MIF₁₋₄₅.

In some embodiments, the MIF-mimic comprises one or more of the peptides selected from Table 2. In some embodiments, the MIF-mimic comprises N- and/or C-terminal chemical modifications to improve ADME-PK. In some embodiments, MIF-mimic comprises non-natural amino acids. In some embodiments, MIF-mimic comprises cyclical variants.

TABLE 2
CTNVPRASVPDGC
CVPRASC
VNTNVPRASVPDGFLSEL
NTNVPRASVPDGFLSEL
TNVPRASVPDGFLSEL
NVPRASVPDGFLSEL
VPRASVPDGFLSEL
PRASVPDGFLSEL
RASVPDGFLSEL
ASVPDGFLSEL
SVPDGFLSEL
VPDGFLSEL
NVPRASVPDGFLSE
NVPRASVPDGFLS
NVPRASVPDGFL
NVPRASVPDGF
NVPRASVPDG
NVPRASVPD
NVPRASVP
VPRASVP
PRASVP
VPRASVPDGFL
VPRASVPDGF
VPRASVPDG
VPRASVPD
VPRASVP
VPRAS
MPMFIVNTNVPRASVPDGFLSEC
MPMFIVNTNVPRASV
FIVNTNVPRASVPDG
NTNVPRASVPDGFLS
VPRASVPDGFLSELT

In some embodiments, the MIF-mimic adopts structural or functional features similar to the amino acid residues 65-94 (numbering includes the first methionine). In some embodiments, the MIF-mimic is a peptide. In some embodiments, the MIF-mimic comprises a peptide of Formula (III):

I/L-G-X¹-X²-X³-X⁴-X⁵-X⁶-N-X⁷-X⁸-X⁹-X¹⁰-X¹¹-X¹²-L/I-X¹³-X¹⁴-X¹⁵-X¹⁶-X¹⁷-X¹⁸-X¹⁹-L/V—X²⁰-I-X²¹-X²²-X²³-X²⁴

wherein:
X¹ is selected from the group consisting of lysine, arginine, cysteine, serine and alanine;
X² is selected from the group consisting of isoleucine, valine and phenylalanine;
X³ is selected from the group consisting of glycine, asparagine and serine;
X⁴ is selected from the group consisting of glycine, proline, alanine, aspartic acid and glutamic acid;
X⁵ is selected from the group consisting of alanine, proline, lysine, arginine, asparagine, aspartic acid and glutamic acid;
X⁶ is selected from the group consisting of glutamine, valine, lysine, arginine, leucine, aspartic acid and glutamic acid;
X⁷ is selected from the group consisting of lysine, arginine, asparagine, isoleucine and valine;
X⁸ is selected from the group consisting of serine, asparagine, glutamine, aspartic acid, glutamic acid, lysine and arginine;
X⁹ is selected from the group consisting of tyrosine, histidine and asparagine;
X¹⁰ is selected from the group consisting of serine, threonine and alanine;
X¹¹ is selected from the group consisting of lysine, aspartic acid, glutamic acid, alanine, serine and glycine;
X¹² is selected from the group consisting of leucine, glutamine, lysine, arginine, leucine, serine and alanine;
X¹³ is selected from the group consisting of cysteine, tyrosine, phenylalanine, serine, alanine and threonine;
X¹⁴ is selected from the group consisting of glycine, aspartic acid, glutamic acid, lysine and arginine;
X¹⁵ is selected from the group consisting of leucine, glutamine, isoleucine, histidine and phenylalanine;
X¹⁶ is selected from the group consisting of leucine, methionine, isoleucine and cysteine;
X¹⁷ is selected from the group consisting of alanine, threonine, serine, arginine, lysine, alanine, glutamine and glycine;
X¹⁸ is selected from the group consisting of glutamic acid, aspartic acid, lysine and arginine;
X¹⁹ is selected from the group consisting of arginine, histidine, glutamine, aspartic acid, glutamic acid, glycine, threonine and lysine;
X²⁰ is selected from the group consisting of arginine, histidine, glycine, asparagine, lysine, arginine, aspartic acid and glutamic acid;
X²¹ is selected from the group consisting of serine, aspartic acid, glutamic acid, lysine, arginine and proline;
X²² is selected from the group consisting of proline, alanine, lysine, arginine and glycine;
X²³ is selected from the group consisting of aspartic acid, glutamic acid, asparagine and alanine; and
X²⁴ is selected from the group consisting of histidine, tyrosine, lysine and arginine.

In some embodiments, X¹ is lysine. In some embodiments, X² is isoleucine. In some embodiments, X³ is glycine. In some embodiments, X⁴ is glycine. In some embodiments, X⁵ is alanine. In some embodiments, X⁶ is glutamine. In some embodiments, X⁷ is arginine. In some embodiments, X⁸ is serine. In some embodiments, X⁹ is tyrosine. In some embodiments, X¹⁰ is serine. In some embodiments, X¹¹ is lysine. In some embodiments, X¹² is leucine. In some embodiments, X¹³ is cysteine. In some embodiments, X¹⁴ is glycine. In some embodiments, X¹⁵ is leucine. In some embodiments, X¹⁶ is leucine. In some embodiments, X¹⁷ is alanine. In some embodiments, X¹⁸ is glutamic acid. In some embodiments, X¹⁹ is arginine. In some embodiments, X²⁰ is arginine. In some embodiments, X²¹ is serine. In some embodiments, X²² is proline. In some embodiments, X²³ is aspartic acid. In some embodiments, X²⁴ is arginine.

In some embodiments, the MIF-mimic comprises any 5 or more consecutive peptide of Formula (III).

In some embodiments, the MIF-mimic comprises 5 or more consecutive amino acids of human MIF₆₅₋₉₄. In some embodiments, the MIF-mimic comprises 5 or more consecutive amino acids of murine MIF₆₅₋₉₄. In some embodiments, the MIF-mimic comprises 5 or more consecutive amino acids of porcine MIF₆₅₋₉₄. In some embodiments, the MIF-mimic comprises 5 or more consecutive amino acids of bovine MIF₆₅₋₉₄. In some embodiments, the MIF-mimic comprises 5 or more consecutive amino acids of rat MIF₆₅₋₉₄.

In some embodiments, the MIF-mimic comprises one or more of the peptides selected from Table 3. In some embodiments, the MIF-mimic comprises N- and/or C-terminal chemical modifications to improve ADME-PK. In some embodiments, MIF-mimic comprises non-natural amino acids. In some embodiments, MIF-mimic comprises cyclical variants.

TABLE 3
CSLHSIGKIGGAQNR
IGKIGGAQNRSYSKL
HSIGKIGGAQNRSYSKLLCGLL
HSIGKIGGAQNRSYSKLLCG
HSIGKIGGAQNRSYSKLL
HSIGKIGGAQNRSYSK
HSIGKIGGAQNRSYS
IGKIGGAQNRSYSKLLC
KIGGAQNRSYSKLLC
GGAQNRSYSKLLCGLLAERLRI
AQNRSYSKLLCGLLAERLRI
NRSYSKLLCGLLAERLRI
SYSKLLCGLLAERLRI
YSKLLCGLLAERLRI
GAQNRSYSKLLCGLLAE
GAQNRSYSKLLCGLL
QNRSYSKLLCGLLAE
HSIGKIGGAQNRSY
HSIGKIGGAQNR
HSIGKIGGAQNRSYSK
IGKIGGAQNRSYSKLLC
KIGGAQNRSYSKLLC
KIGGAQNRSYS
GAQNRSYSKLLCGLLAE
GAQNRSYSKLLCGLL
GAQNRSYSKLLCG
GAQNRSYSKLL
QNRSYSKLLCGLLAE
RSYSKLLCGLLAE
YSKLLCGLLAE
IAVHVVPDQLMAFGGSSEPCALCSLHSIGKIGGAQNRSYSKLL
IAVHVVPDQLMAFGGSSEPCALCSLHSIGKIGGAQNRSY
IAVHVVPDQLMAFGGSSEPCALCSLHSIGKIGGAQ
IAVHVVPDQLMAFGGSSEPCALCSLHSIGKI
IAVHVVPDQLMAFGGSSEPCALCSLHS
IAVHVVPDQLMAFGGSSEPCALC
IAVHVVPDQLMAFGGSSEP
IAVHVVPDQLMAFGG
IAVHVVPDQLM
IAVHVVPDQLMAFGGSSEPCALCSLHSIGKIGGAQNRSYSKLL
VVPDQLMAFGGSSEPCALCSLHSIGKIGGAQNRSYSKLL
QLMAFGGSSEPCALCSLHSIGKIGGAQNRSYSKLL
FGGSSEPCALCSLHSIGKIGGAQNRSYSKLL
SEPCALCSLHSIGKIGGAQNRSYSKLL
ALCSLHSIGKIGGAQNRSYSKLL
LHSIGKIGGAQNRSYSKLL
GKIGGAQNRSYSKLL
IGGAQNRSYSKLL
QNRSYSKLL
IGKIGGAQNRSYSKL
IGKIGGAQ
linear (CIGKIGGAQC)
cyclo (CIGKIGGAQC)
RSYSKLLCGLLAE
linear (CRSYSKLLCGLLAEC)
cyclo (CRSYSKLLCGLLAEC)
CGLLAERLRISPDR
linear(CGLLAERLRISPDRC)
Cyclo (CGLLAERLRISPDRC

### E. CD74 Mimics

In some embodiments, the modulator of MIF is an agent that disrupts the ability of CD74 to form a signaling complex with CXCR2, CXCR4, MIF, or a combination thereof. In some embodiments, the modulator of MIF is a CD74-like peptide that mimics part or all of a CD74 domain (e.g., the C-terminal/extracellular (lumenal) domain). In some embodiments, the CD74-mimic binds to MIF, CXCR2, and/or CXCR4 and thus prevents CD74 from binding to MIF, CXCR2, and/or CXCR4.

In some embodiments, the CD74-mimic adopts structural or functional features similar to CD74. In some embodiments, the CD74-mimic is a peptide.

In some embodiments, the CD74-mimic comprises 5 or more consecutive amino acids of human CD74. In some embodiments, the CD74-mimic comprises 5 or more consecutive amino acids of bovine CD74. In some embodiments, the CD74-mimic comprises 5 or more consecutive amino acids of porcine CD74. In some embodiments, the CD74-mimic comprises 5 or more consecutive amino acids of murine CD74. In some embodiments, the CD74-mimic comprises 5 or more consecutive amino acids of rat CD74.

In some embodiments, the CD74-mimic comprises one or more of the peptides selected from Table 3. In some embodiments, the CD-74-mimic comprises N- and/or C-terminal chemical modifications to improve ADME-PK. In some embodiments, CD74-mimic comprises non-natural amino acids. In some embodiments, CD74-mimic comprises cyclical variants.

TABLE 4

| | |
|---|---|
| AYFLYQQQ | TKYGNMTEDHVMHLL |
| QQQGRLDKLTVTGRL | HVMHLLQNADPLKVY |
| GRLDKLTVTSQNLQL | DPLKVYPPLKGSFPE |
| SQNLQLENLRM | KGSFPENLRHLKNTM |
| TVTGRLDKLTVTSQN | HLKNTMETIDWKVFE |
| TVTSQNLQLENLRM | DWKVFESWMHHWLLF |
| LENLRMKLPKPPKPV | HHWLLFEMSRHSLEQ |
| KLPKPPKPVSKMRMA | RHSLEQKPTDAPPKE |
| SKMRMATPL | DAPPKESLELEDPSS |
| LMQALPMGALPQGPM | LEDPSSGLGVTKQDL |
| LPQGPMQNATKYGNM | VTKQDLGPVPM |

### E. CXCR2/CXCR4 Mimics

In some embodiments, the modulator of MIF is an agent that disrupts the ability of CXCR2 and/or CXCR4 to form a signaling complex with CD74 and/or MIF.

In some embodiments, the modulator of MIF is a CXCR2-like peptide that mimics part or all of a CXCR2 domain. In some embodiments, the modulator of MIF is a CXCR2-like peptide that mimics part or all of the CXCR2 extracellular loop 1 and/or extracellular loop 2. In some embodiments, the CXCR2-mimic binds to MIF and/or CD74 and thus prevents CXCR2 from binding to MIF and/or CD74.

In some embodiments, the modulator of MIF is a CXCR4-like peptide that mimics part or all of a CXCR4 domain. In some embodiments, the modulator of MIF is a CXCR4-like peptide that mimics part or all of the CXCR4 extracellular loop 1 and/or extracellular loop 2. In some embodiments, the modulator of MIF is a CXCR4-like peptide that mimics part or all of the CXCR4 amino acids 182-202 (SEADDRYICDRFYPNDLWVVV). In some embodiments the modulator of MIF is a CXCR4-like peptide that mimics part or all of the CXCR4 amino acids 185-199 (DDRYICDRFYPNDLW). In some embodiments, the CXCR4-mimic binds to MIF and/or CD74 and thus prevents CXCR4 from binding to MIF and/or CD74.

In some embodiments, the CXCR4-mimic or the CXCR2 mimic comprises one or more of the peptides selected from Table 4. In some embodiments, the mimic comprises N- and/or C-terminal chemical modifications to improve ADME-PK. In some embodiments, the mimic comprises non-natural amino acids. In some embodiments, mimic comprises cyclical variants.

TABLE 5
DLSNYSYSSTLPPFL
DLSNYSYSSTLPP
DLSNYSYSSTL
DLSNYSYSS
DLSNYSY
DLSNY
KVNGWIFGTFL
KVNGWIFGT
KVNGWIF
KVNGW
RRTVYSSNVSPAC
RRTVYSSNVSP
RRTVYSSNV
RRTVYSS
RRTVY
EDMGNNTANWRML
EDMGNNTANWR
EDMGNNTAN
EDMGNNT
EDMGN
MRTQVIQETCERR
MRTQVIQETCE
MRTQVIQET
MRTQVIQ
MRTQV
CERRNHIDRALDA
CERRNHIDRAL
CERRNHIDR
CERRNHI
CERRN
DRYICDRFYPNDL
DRYICDRFYPN
DRYICDRFY
DRYICDR
DRYIC
ICDRFYPNDLWVV
ICDRFYP
ICDRF
RFYPNDLWVVVFQ
RFYPNDLWVVV
RFYPNDLWV
RFYPNDL
RFYPN

### F. Fusion Peptide

In some embodiments, the modulator of MIF is an agent that disrupts the ability of MIF to form a signaling complex with CXCR2, CXCR4, CD74, or a combination thereof. In some embodiments, the modulator of MIF is a fusion peptide that binds both the N-loop domain of MIF and the pseudo-ELR domain of MIF.

In some embodiments, the peptides that comprise the fusion peptide are derived from human MIF, bovine MIF, porcine MIF, murine MIF, rat MIF, or a combination thereof. In some embodiments, the peptides that comprise the fusion peptide are artificially constructed.

In some embodiments, the fusion peptide comprises at least one peptide that adopts structural or functional features similar to the N-loop motif of MIF, and at least one peptide that adopts structural or functional features similar to the pseudo-ELR loop of MIF. In some embodiments, the fusion peptide comprises (a) a first peptide that adopts structural or functional features similar to the N-loop motif of MIF; and (b) a second peptide that adopts structural or functional features similar to the pseudo-ELR loop of MIF. In some embodiments, the fusion peptide comprise (a) a first peptide that adopts structural or functional features similar to the N-loop motif of MIF; (b) a second peptide that adopts structural or functional features similar to a first portion of the pseudo-ELR loop of MIF; and (c) a third peptide that adopts structural or functional features similar to a second portion of the pseudo-ELR loop of MIF.

In some embodiments, the fusion peptide comprise (a) a first peptide that adopts structural or functional features similar to the N-loop motif of MIF; and (b) a second peptide that adopts structural or functional features similar to the pseudo-ELR loop of MIF; wherein the first peptide and the second peptide are chemically linked. In some embodiments, the fusion peptide comprise (a) a first peptide that adopts structural or functional features similar to the N-loop motif of MIF; (b) a second peptide that adopts structural or functional features similar to a first portion of the pseudo-ELR loop of MIF; and (c) a third peptide that adopts structural or functional features similar to a second portion of the pseudo-ELR loop of MIF; wherein the first peptide, the second peptide, and the third peptide are chemically linked.

In some embodiments, the fusion peptide comprises (a) a first peptide having the sequence MAFGGSSEPC; and (b) a second peptide having the sequence NVPRA. In some embodiments, the fusion peptide comprises (a) a first peptide having the sequence MAFGGSSEPC; (b) a second peptide having the sequence NVPRA; and (c) a third peptide having the sequence SVPDG.

In some embodiments, the methods and compositions disclosed herein comprise (a) a first peptide having the sequence LQDP; and (b) a second peptide having the sequence NVPRA.

In some embodiments, the first peptide and the second peptide are directly bound to each other (e.g., via a covalent or ionic bond).

### Linkers

In some embodiments, at least one peptide that adopts structural or functional features similar to the N-loop motif of MIF and at least one peptide that adopts structural or functional features similar to the pseudo-ELR loop of MIF are indirectly bound to each other (e.g., via a linker). In some embodiments, at least one peptide that adopts structural or functional features similar to the N-loop motif of MIF and at least one peptide that adopts structural or functional features similar to the pseudo-ELR loop of MIF are bound by a linker.

In some embodiments, the linker binds (a) a first peptide that adopts structural or functional features similar to the N-loop motif of MIF; and (b) a second peptide that adopts structural or functional features similar to the pseudo-ELR loop of MIF. In some embodiments, the fusion peptide is a peptide of Formula (IV):

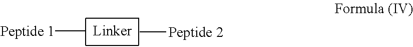

wherein Peptide 1, and Peptide 2 are selected from any peptide disclosed herein.

In some embodiments, the linker binds (a) a first peptide that adopts structural or functional features similar to the N-loop motif of MIF; (b) a second peptide that adopts structural or functional features similar to a first portion of the pseudo-ELR loop of MIF; and (c) a third peptide that adopts structural or functional features similar to a second portion of the pseudo-ELR loop of MIF. In some embodiments, the fusion peptide is a peptide of Formula (V):

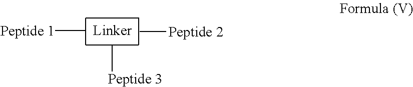

wherein Peptide 1, Peptide 2, and Peptide 3 are selected from any peptide disclosed herein.

As used herein, a “linker” is any molecule capable of binding (e.g., covalently) to multiple peptides. In some embodiments, the linker binds to the peptide by a covalent linkage. In some embodiments, the covalent linkage comprises a ether bond, thioether bond, amine bond, amide bond, carbon-carbon bond, carbon-nitrogen bond, carbon-oxygen bond, or carbon-sulfur bond.

In some embodiments, the linker is flexible. In some embodiments, the linker is rigid. In some embodiments, the linker is long enough to allow the fusion peptide to bind to both the pseudo-ELR and N-loop domains of MIF.

In some embodiments, the linker binds to two peptides. In some embodiments, the linker binds to three peptides.

In some embodiments, a linker described herein binds to the C-terminus of one or more of the peptides that form the fusion peptide. In some embodiments, the linker binds to the N-terminus of one or more of the peptides that form the fusion peptide. In some embodiments, a linker described herein binds to the C-terminus of one or more of the peptides and the N-terminus of any remaining peptides.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker is an alkyl. In some embodiments, the linker is heteroalkyl.

In some embodiments, the linker is an alkylene. In some embodiments, the linker is an alkenylene. In some embodiments, the linker is an alkynylene. In some embodiments, the linker is a heteroalkylene.

An “alkyl” group refers to an aliphatic hydrocarbon group. The alkyl moiety may be a saturated alkyl or an unsaturated alkyl. Depending on the structure, an alkyl group can be a monoradical or a diradical (i.e., an alkylene group).

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

In some embodiments, the linker comprises a ring structure (e.g., an aryl). As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Cycloalkyls include, but are not limited to, cyclopropyl, cyclobutyl, cyclopentyl, cyclohexyl, cycloheptyl, and cyclooctyl.

In some embodiments, the ring is a cycloalkane. In some embodiments, the ring is a cycloalkene.

In some embodiments, the ring is an aromatic ring. The term “aromatic” refers to a planar ring having a delocalized π-electron system containing 4n+2π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

In some embodiments, the ring is a heterocycle. The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

In some embodiments, the ring is fused. The term “fused” refers to structures in which two or more rings share one or more bonds. In some embodiments, the ring is a dimer. In some embodiments, the ring is a trimer. In some embodiments, the ring is a substituted.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

In some embodiments, the linker is substituted. The term “optionally substituted” or “substituted” means that the referenced group may be substituted with one or more additional group(s) individually and independently selected from C₁-C₆alkyl, C₃-C₈cycloalkyl, aryl, heteroaryl, C₂-C₆heteroalicyclic, hydroxy, C₁-C₆alkoxy, aryloxy, C₁-C₆alkylthio, arylthio, C₁-C₆alkylsulfoxide, arylsulfoxide, C₁-C₆alkylsulfone, arylsulfone, cyano, halo, C₂-C₈acyl, C₂-C₈acyloxy, nitro, C₁-C₆haloalkyl, C₁-C₆-fluoroalkyl, and amino, including C₁-C₆alkylamino, and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each R^{s} is independently selected from H, (C₁-C₄alkyl), (C₃-C₈cycloalkyl), heteroaryl, aryl, and C₁-C₆heteroalkyl. Optionally substituted non-aromatic groups may be substituted with one or more oxo (═O). The protecting groups that may form the protective derivatives of the above substituents are known to those of skill in the art.

In some embodiments, the linker is an amino acid. In some embodiments, the fusion peptide is a peptide of Formula (VI):

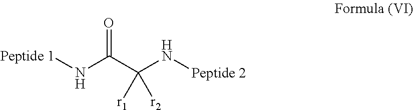

wherein Peptide 1, and Peptide 2 are selected from any peptide disclosed herein.

In some embodiments, the linker is an artificial amino acid. In some embodiments, the linker is a β-amino acid. In some embodiments, the linker is a γ-amino acid.

In some embodiments, the linker is a polyethylene glycol (PEG). In some embodiments, the linker is a diamino acid. In some embodiments, the linker is diaminopropionic acid.

In some embodiments, the linker is hydrolyzible.

By way of non-limiting example, the fusion peptide is:

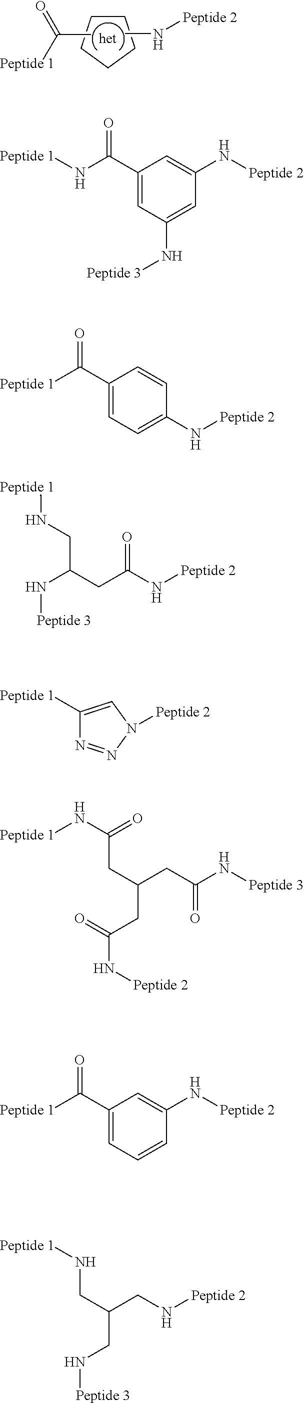

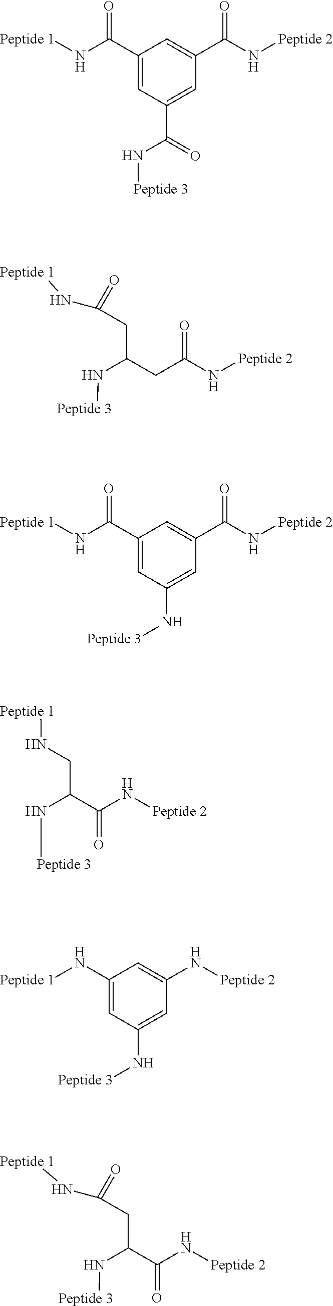

wherein Peptide 1, Peptide 2, and Peptide 3 are selected from any peptide disclosed herein.

### E. MIF Trimerization Modulating Agents

In some embodiments, the modulator of MIF is an agent that modulates the ability of MIF to form a homo-multimer. In some embodiments, the modulator of MIF is an agent that disrupts the ability of MIF to form a trimer. In some embodiments, an inflammatory disease, disorder, condition, or symptom is treated by promoting MIF trimerization.

In certain instances, functionally-active MIF comprises three MIF peptide sequences (i.e., a trimer). In certain instances, the pseudo-ELR loops of each MIF polypeptide form a ring in the trimer. In certain instances, the N-loop motifs of each MIF polypeptide extend outwards from the pseudo-ELR ring. In certain instances, disruption of the trimer disrupts the high affinity binding of MIF to its target receptors.

In certain instances, residues 38-44 of one subunit interact with residues 48-50 of a second subunit. In certain instances, residues 96-102 of one subunit interact with residues 107-109 of a second subunit. In certain instances, a domain on one subunit formed by N73 R74S77 K78 C81 (numbering includes the first methionine) interacts with N110 S111 T112 (numbering includes the first methionine) of a second subunit.

In some embodiments, a MIF trimerization disrupting agent is derived from and/or incorporates any or all of amino acid residues 38-44 of MIF (e.g., human, bovine, procine, murine, or rat). In some embodiments, a MIF trimerization disrupting agent is a peptide derived from and/or incorporates any or all of amino acid residues 48-50 of MIF (e.g., human, bovine, procine, murine, or rat). In some embodiments, a MIF trimerization disrupting agent is a peptide derived from and/or incorporates any or all of amino acid residues 57-66 of MIF (e.g., human, bovine, procine, murine, or rat). In some embodiments, a MIF trimerization disrupting agent is a peptide derived from and/or incorporates any or all of amino acid residues 61-70 of MIF (e.g., human, bovine, procine, murine, or rat). In some embodiments, a MIF trimerization disrupting agent is a peptide derived from and/or incorporates any or all of amino acid residues 96-102 of MIF (e.g., human, bovine, procine, murine, or rat). In some embodiments, a MIF trimerization disrupting agent is a peptide derived from and/or incorporates any or all of amino acid residues 107-109 of MIF (e.g., human, bovine, procine, murine, or rat). In some embodiments, a MIF trimerization disrupting agent is a peptide derived from and/or incorporates any or all of amino acid residues N73, R74, S77, K78, and C81 of MIF (e.g., human, bovine, procine, murine, or rat) (numbering includes the first methionine). In some embodiments, a MIF trimerization disrupting agent is a peptide derived from and/or incorporates any or all of amino acid residues N110, S111, and T112 of MIF (e.g., human, bovine, procine, murine, or rat) (numbering includes the first methionine).

In some embodiments, a MIF trimerization disrupting agent is a peptide derived from and/or incorporates any or all of amino acid residues 57-66 of MIF (numbering includes the first methionine). In some embodiments, a MIF trimerization disrupting agent is a peptide of Formula (VII):

X¹-X²-X³-X⁴-X⁵-X⁶-X⁷-S/A-I-G

wherein:
X¹ is selected from the group consisting of cysteine, alanine, serine, and threonine;
X² is selected from the group consisting of alanine, proline, glycine and cysteine;
X³ is selected from the group consisting of leucine, valine and pheynylalanine;
X⁴ is selected from the group consisting of cysteine, glycine, threonine and isoleucine;
X⁵ is selected from the group consisting of serine, valine, glutamine and asparagine;
X⁶ is selected from the group consisting of leucine, valine, isoleucine and methionine; and
X⁷ is selected from the group consisting of histidine, cysteine, lysine, arginine, and leucine.

In some embodiments, X¹ is. In some embodiments, X² is. In some embodiments, X³ is. In some embodiments, X⁴ is. In some embodiments, X⁵ is. In some embodiments, X⁶ is. In some embodiments, X⁷ is.

In some embodiments, the MIF-mimic comprises any 5 or more consecutive peptide of Formula (VIII).

In some embodiments, the MIF-mimic comprises 5 or more consecutive amino acids of human MIF₅₇₋₆₆. In some embodiments, the MIF-mimic comprises 5 or more consecutive amino acids of murine MIF₅₇₋₆₆. In some embodiments, the MIF-mimic comprises 5 or more consecutive amino acids of porcine MIF₅₇₋₆₆. In some embodiments, the MIF-mimic comprises 5 or more consecutive amino acids of bovine MIF₅₇₋₆₆. In some embodiments, the MIF-mimic comprises 5 or more consecutive amino acids of rat MIF₅₇₋₆₆.

In some embodiments, a MIF trimerization disrupting agent is an antibody that binds to any or all of amino acid residues 38-44 of MIF. In some embodiments, a MIF trimerization disrupting agent is an antibody that binds to any or all of amino acid residues 48-50 of MIF. In some embodiments, a MIF trimerization disrupting agent is an antibody that binds to any or all of amino acid residues 57-66 of MIF. In some embodiments, a MIF trimerization disrupting agent is an antibody that binds to any or all of amino acid residues 61-70 of MIF. In some embodiments, a MIF trimerization disrupting agent is an antibody that binds to any or all of amino acid residues 96-102 of MIF. In some embodiments, a MIF trimerization disrupting agent is an antibody that binds to any or all of amino acid residues 107-109 of MIF. In some embodiments, a MIF trimerization disrupting agent is an antibody that binds to any or all of amino acid residues N73, R74, S77, K78, and C81 of MIF. In some embodiments, a MIF trimerization disrupting agent is an antibody that binds to any or all of amino acid residues N110, S111, and T112 of MIF.

In some embodiments, a MIF trimerization disrupting agent is a small molecule that binds to any or all of amino acid residues 38-44 of MIF. In some embodiments, a MIF trimerization disrupting agent is a small molecule that binds to any or all of amino acid residues 48-50 of MIF. In some embodiments, a MIF trimerization disrupting agent is a small molecule that binds to any or all of amino acid residues 57-66 of MIF. In some embodiments, a MIF trimerization disrupting agent is a small molecule that binds to any or all of amino acid residues 61-70 of MIF. In some embodiments, a MIF trimerization disrupting agent is a small molecule that binds to any or all of amino acid residues 96-102 of MIF. In some embodiments, a MIF trimerization disrupting agent is a small molecule that binds to any or all of amino acid residues 107-109 of MIF. In some embodiments, a MIF trimerization disrupting agent is a small molecule that binds to any or all of amino acid residues N73, R74, S77, K78, and C81 of MIF. In some embodiments, a MIF trimerization disrupting agent is a small molecule that binds to any or all of amino acid residues N110, S111, and T112 of MIF.

In some embodiments, a MIF trimerization disrupting agent is a peptibody that binds to any or all of amino acid residues 38-44 of MIF. In some embodiments, a MIF trimerization disrupting agent is a peptibody that binds to any or all of amino acid residues 48-50 of MIF. In some embodiments, a MIF trimerization disrupting agent is a peptibody that binds to any or all of amino acid residues 57-66 of MIF. In some embodiments, a MIF trimerization disrupting agent is a peptibody that binds to any or all of amino acid residues 61-70 of MIF. In some embodiments, a MIF trimerization disrupting agent is a peptibody that binds to any or all of amino acid residues 96-102 of MIF. In some embodiments, a MIF trimerization disrupting agent is a peptibody that binds to any or all of amino acid residues 107-109 of MIF. In some embodiments, a MIF trimerization disrupting agent is a peptibody that binds to any or all of amino acid residues N73, R74, S77, K78, and C81 of MIF. In some embodiments, a MIF trimerization disrupting agent is a peptibody that binds to any or all of amino acid residues N110, S111, and T112 of MIF.

### F. Peptide Mimetics

In some embodiments, a peptide mimetic is used in place of the peptides described herein, including for use in the treatment or prevention of an inflammatory disorder.

Peptide mimetics (and peptide-based inhibitors) are developed using, for example, computerized molecular modeling. Peptide mimetics are designed to include structures having one or more peptide linkages optionally replaced by a linkage selected from the group consisting of: —CH₂NH—, —CH₂S—, —CH₂—CH₂—, —CH═CH-(cis and trans), —CH═CF-(trans), —CoCH₂—, —CH(OH)CH₂—, and —CH₂SO—, by methods well known in the art. In some embodiments such peptide mimetics have greater chemical stability, enhanced pharmacological properties (half-life, absorption, potency, efficacy, etc.), altered specificity (e.g., a broad-spectrum of biological activities), reduced antigenicity, and are more economically prepared. In some embodiments peptide mimetics include covalent attachment of one or more labels or conjugates, directly or through a spacer (e.g., an amide group), to non-interfering positions(s) on the analog that are predicted by quantitative structure-activity data and/or molecular modeling. Such non-interfering positions generally are positions that do not form direct contacts with the receptor(s) to which the peptide mimetic specifically binds to produce the therapeutic effect. In some embodiments, systematic substitution of one or more amino acids of a consensus sequence with a D-amino acid of the same type (e.g., D-lysine in place of L-lysine) are used to generate more stable peptides with desired properties.

Phage display peptide libraries have emerged as a technique in generating peptide mimetics (Scott, J. K. et al. (1990) Science 249:386; Devlin, J. J. et al. (1990) Science 249:404; U.S. Pat. No. 5,223,409, U.S. Pat. No. 5,733,731; U.S. Pat. No. 5,498,530; U.S. Pat. No. 5,432,018;U.S. Pat. No. 5,338,665; U.S. Pat. No. 5,922,545; WO 96/40987 and WO 98/15833 (each of which is incorporated by reference for such disclosure). In such libraries, random peptide sequences are displayed by fusion with coat proteins of filamentous phage. Typically, the displayed peptides are affinity-eluted against an antibody-immobilized extracellular domain. In some embodiments peptide mimetics are isolated by biopanning (Nowakowski, G. S, et al. (2004) Stem Cells 22:1030-1038). In some embodiments whole cells expressing MIF are used to screen the library utilizing FACs to isolate phage specifically bound cells. The retained phages are enriched by successive rounds of biopanning and repropagation. The best binding peptides are sequenced to identify key residues within one or more structurally related families of peptides. The peptide sequences also suggest which residues to replace by alanine scanning or by mutagenesis at the DNA level. In some embodiments mutagenesis libraries are created and screened to further optimize the sequence of the best binders. Lowman (1997) Ann. Rev. Biophys. Biomol. Struct. 26:401-24.

In some embodiments structural analysis of protein-protein interaction is used to suggest peptides that mimic the binding activity of the polypeptides described herein. In some embodiments the crystal structure resulting from such an analysis suggests the identity and relative orientation of critical residues of the polypeptide, from which a peptide is designed. See, e.g., Takasaki, et al. (1997) Nature Biotech, 15: 1266-70.

In some embodiments, the agent is a peptide or polypeptide. In some embodiments, the peptide is a peptide that mimics a peptide sequence as follows: VNTNVPPRASVPDGFLSELTQQLAQATGKPPQYIAVHVVPDQL and the corresponding feature/domain of at least one of a MIF monomer or MIF trimer; a peptide that mimics a peptide sequence as follows: PDQLMAFGGSSEPCALCSL and the corresponding feature/domain of at least one of a MIF monomer or MIF trimer; a peptide that mimics a peptide sequence as follows: VNTNVPPRASVPDGFLSELTQQLAQATGKPPQYIAVHVVPDQLMAFGGSSEPCALCSL and the corresponding feature/domain of at least one of a MIF monomer or MIF trimer; a peptide that mimics a peptide sequence as follows: PDQLMAFGGSSEPCALCSLHSI and the corresponding feature/domain of at least one of a MIF monomer or MIF trimer; or combinations thereof.

### V. COMBINATIONS

Disclosed herein, in certain embodiments, are methods and pharmaceutical compositions for modulating an inflammatory disorder comprising a synergistic combination of (a) a modulator of MIF; and (b) a second active agent that treats inflammation through an alternative pathway.

Further disclosed herein, in certain embodiments, are methods and compositions for treating inflammatory disorders. In some embodiments, the method comprises co-administering a synergistic combination of (a) a modulator of MIF; and (b) a second active agent selected from an agent that inhibits inflammation and modulates a lipid.

In some embodiments, the combination is synergistic and results in a more efficacious therapy. In some embodiments, therapy synergistically treats inflammatory disorders by targeting multiple pathways that result in (either partially or fully) development of an inflammatory disorder.

Disclosed herein, in certain embodiments, are methods and pharmaceutical compositions for modulating an inflammatory disorder comprising a synergistic combination of (a) a modulator of MIF; and (b) a second active agent that induces unwanted inflammation. In some embodiments, the second active agent is a bisphosphonate and/or a protease inhibitor.

### Pharmaceutical Therapies Comprising a First Anti-Inflamatory and a Second Anti-Inflammatory

Disclosed herein, in certain embodiments, are methods and pharmaceutical compositions for modulating an inflammatory disorder comprising a synergistic combination of (a) a modulator of MIF; and (b) a second active agent that treats inflammation through an alternative pathway.

In some embodiments, the combination is synergistic and results in a more efficacious therapy. In some embodiments, therapy synergistically treats inflammatory disorders by targeting multiple pathways that result in (either partially or fully) development of an inflammatory disorder.

In some embodiments, the modulator of MIF and a second anti-inflammatory agent, (e.g., an immunosuppressant) synergistically treat an inflammatory disorder by (1) decreasing the chemotaxis of leukocytes, and (2) reducing the influx of cytokines.

In some embodiments, the second anti-inflammatory agent is: cyclosporine A, alefacept, efalizumab, methotrexate, acitretin, isotretinoin, hydroxyurea, mycophenolate mofetil (MMF), sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), ), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Actos® (Pioglitazone), Avandia®(Rosiglitazone), Amaryl® (Glimepiride), Sulfonylurea-types, Diabeta® (Glyburide), Diabinese® (Chlorpropamide), Glucotrol® (Glipizide), Glynasec (glyburide), Micronase® (glyburide), Orinase® (Tolbutamide), Tolinase® (Tolazamide), Glucophage, Riomet® (Metformin), Glucovance® (glyburide+metformin), Avandamet® (Rosiglitazone+metformin), Avandaryl® (Rosiglitazone+glimepiride), Byetta® (Exenatide), Insulins, Januvia® (Sitagliptin), Metaglip® (glipizide and metformin), Prandin® (Repaglinide), Precose® (Acarbose), Starlix® (Nateglinide), Xenical® (Orlistat), cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-C5 antibody (e.g., eculizumab); or combinations thereof.

In certain instances, administration of a 5-ASA causes (either partially or fully) inflammation. In certain instances, administration of sulfasalazine results in (either partially or fully) pneumonitis with or without eosinophilia, vasculitis, pericarditis with or without tamponade, hepatitis, allergic myocarditis, pancreatitis, nephritis, exfoliative dermatitis, serum vasculitis, and/or pleuritis. In certain instances, administration of mesalamine results in (either partially or fully) pericarditis, myocarditis, pancreatitis, hepatitis, interstitial pneumonitis, pleuritis, interstitial nephritis, and/or pneumonitis. In certain instances, administration of olsalazine results in (either partially or fully) myocarditis, pericarditis, pancreatitis, interstitial and/or nephritis.

In some embodiments, the modulator of MIF and a 5-ASA treat an inflammatory disorder by (1) decreasing the chemotaxis of leukocytes, and (2) reducing the synthesis of eicosanoids and inflammatory cytokines. In some embodiments, the modulator of MIF also decreases any undesired inflammation (e.g., pancreatitis) resulting from administration of the 5-ASA.

In some embodiments, the modulator of MIF and an anti-TNF agent treat an inflammatory disorder by (1) decreasing the chemotaxis of leukocytes, and (2) suppressing a TNF-induced cytokine cascade. In some embodiments, the modulator of MIF also decreases any undesired inflammation (e.g., tuberculosis) resulting from administration of the anti-TNF agent.

In some embodiments, the modulator of MIF and a leukotriene inhibitor treat an inflammatory disorder by (1) decreasing the chemotaxis of leukocytes, and (2) antagonizing LTA4, LTB4, LTC4, LTD4, LTE4, LTF4, LTA4R; LTB4R; LTB4R1, LTB4R2, LTC4R, LTD4R, LTE4R, CYSLTR1, or CYSLTR2; or inhibiting the synthesis of a leukotriene via 5-LO, FLAP, LTA4H, LTA4S, or LTC4S. In some embodiments, the modulator of MIF also decreases any undesired inflammation (e.g., tuberculosis) resulting from administration of the leukotriene inhibitor.

In some embodiments, the modulator of MIF and an IL-1 receptor antagonist treat an inflammatory disorder by (1) decreasing the chemotaxis of leukocytes, and (2) blocking the stimulation of T cell IL-1 receptor. In some embodiments, the modulator of MIF also decreases any undesired inflammation (e.g., pneumonia, and bone and joint infections) resulting from administration of the IL-1 receptor antagonist.

In some embodiments, the modulator of MIF and an IL-2 receptor antagonist treat an inflammatory disorder by (1) decreasing the chemotaxis of leukocytes, and (2) blocking the stimulation of T cell IL-2 receptor. In some embodiments, the modulator of MIF also decreases any undesired inflammation (e.g., gastrointestinal disorders) resulting from administration of the IL-2 receptor antagonist.

In some embodiments, the modulator of MIF and a cytotoxic agent treat an inflammatory disorder by (1) decreasing the chemotaxis of leukocytes, and (2) treating neoplastic disease. In some embodiments, the modulator of MIF also decreases any undesired inflammation (e.g., neutropenia) resulting from administration of the cytotoxic agent.

In some embodiments, the modulator of MIF and an immunomodulatory agent treat an inflammatory disorder by (1) decreasing the chemotaxis of leukocytes, and (2) enhancing, or suppressing the immune system. In some embodiments, the modulator of MIF also decreases any undesired inflammation (e.g., hematologic side effects) resulting from administration of the immunomodulatory agent.

In some embodiments, the modulator of MIF and an antibiotic treat an inflammatory disorder by (1) decreasing the chemotaxis of leukocytes, and (2) by blocking cell and/or microbial growth by disrupting the cell cycle, or by blocking histone deacetylase. In some embodiments, the modulator of MIF also decreases any undesired inflammation (e.g., cardiotoxicity) resulting from administration of the antibiotic.

In some embodiments, the modulator of MIF and a T-cell co-stimulatory blocker treat an inflammatory disorder by (1) decreasing the chemotaxis of leukocytes, and (2) modulating a co-stimulatory signal which is required for full T-cell activation. In some embodiments, the modulator of MIF also decreases any undesired inflammation (e.g., neutropenia) resulting from administration of the T-cell co-stimulatory blocker.

In some embodiments, the modulator of MIF and a B cell depleting agent treat an inflammatory disorder by (1) decreasing the chemotaxis of leukocytes, and (2) inhibits B-cell activity. In some embodiments, the modulator of MIF also decreases any undesired inflammation (e.g., Progressive Multifocal Leukoencephalopathy) resulting from administration of the B-cell depleting agent.

In some embodiments, the modulator of MIF and an immunosuppressive agent treat an inflammatory disorder by (1) decreasing the chemotaxis of leukocytes, and (2) selectively or non-selectively inhibits or prevents activity of the immune system. In some embodiments, the modulator of MIF also decreases any undesired inflammation (e.g., lymphoma) resulting from administration of immunosuppressive agent.

In some embodiments, the modulator of MIF and an alkylating agent treat an inflammatory disorder by (1) decreasing the chemotaxis of leukocytes, and (2) inducing covalent binding of alkyl groups to cellular molecules. In some embodiments, the modulator of MIF also decreases any undesired inflammation (e.g., immune suppression) resulting from administration of the alkylating agent.

In some embodiments, the modulator of MIF and an anti-metabolite treat an inflammatory disorder by (1) decreasing the chemotaxis of leukocytes, and (2) preventing the biosynthesis or use of normal cellular metabolites. In some embodiments, the modulator of MIF also decreases any undesired inflammation (e.g., mutagenesis) resulting from administration of the anti metabolite.

In some embodiments, the modulator of MIF and a plant alkaloid treat an inflammatory disorder by (1) decreasing the chemotaxis of leukocytes, and (2) interfering with normal microtubule breakdown during cell division. In some embodiments, the modulator of MIF also decreases any undesired inflammation (e.g., leukopenia) resulting from administration of the plant alkaloid.

In some embodiments, the modulator of MIF and a terpenoid treat an inflammatory disorder by (1) decreasing the chemotaxis of leukocytes, and (2) treating neoplastic disease or microbial infections. In some embodiments, the modulator of MIF also decreases any undesired inflammation resulting from administration of the terpenoid agent.

In some embodiments, the modulator of MIF and a topoisomerase inhibitor treat an inflammatory disorder by (1) decreasing the chemotaxis of leukocytes, and (2) modulating the action of cellular topoisomerase enzymes. In some embodiments, the modulator of MIF also decreases any undesired inflammation (e.g., gastrointestinal effects) resulting from administration of the topoisomerase inhibitor.

In some embodiments, the modulator of MIF and an antibody treat an inflammatory disorder by (1) decreasing the chemotaxis of leukocytes, and (2) neutralizing inflammatory cytokines such as, for example, TNF alpha. In some embodiments, the modulator of MIF also decreases any undesired inflammation (e.g., tuberculosis) resulting from administration of the antibody.

In some embodiments, the modulator of MIF and a hormonal therapy treat an inflammatory disorder by (1) decreasing the chemotaxis of leukocytes, and (2) suppressing cytokine release. In some embodiments, the modulator of MIF also decreases any undesired inflammation (e.g., cancer) resulting from administration of the hormone.

In some embodiments, the modulator of MIF and an anti-diabetes therapy treat an inflammatory disorder by (1) decreasing the chemotaxis of leukocytes, and (2) improving sensitivity to insulin in muscle and adipose tissue. In some embodiments, the modulator of MIF also decreases any undesired inflammation (e.g., liver inflammation, pancreatitis) resulting from administration of the anti-diabetes agent.

### Pharmaceutical Therapies Comprising an Anti-Inflammatory and a Lipid Modulator

Disclosed herein, in certain embodiments, are methods and compositions for treating inflammatory disorders. In some embodiments, the method comprises co-administering a synergistic combination of (a) a modulator of MIF; and (b) a second active agent selected from an agent that modulates a lipid and induces undesired inflammation.

In some embodiments, therapy synergistically treats inflammatory disorders by (a) targeting multiple pathways that result in (either partially or fully) development of an inflammatory disorder and (b) treating and/or ameliorating undesired inflammation (e.g., myositis) resulting from the second anti-inflammatory agent disorder agent.

In some embodiments, the modulator of a lipid and/or a lipoprotein selectively increases the levels of ApoA-I protein (e.g. by transcriptional induction of the gene encoding ApoA-I) and increases the production of nascent HDL (ApoAI-enriched). In some embodiments, the modulator of a lipid and/or a lipoprotein is DF4 (Ac-D-W-F-K-A-F-Y-D-K-V-A-E-K-F-K-E-A-F—NH2); DF5; RVX-208 (Resverlogix); or combinations thereof.

In some embodiments, the modulator of a lipid and/or a lipoprotein is avasimibe; pactimibe sulfate (CS-505); CI-1011 (2,6-diisopropylphenyl[(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl) urea); HL-004 (N-(2,6-diisopropylphenyl) tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof.

In some embodiments, the modulator of a lipid and/or a lipoprotein (partially or completely) inhibits the activity of Cholesteryl Ester Transfer Protein (CETP). In some embodiments, the modulator of a lipid and/or a lipoprotein is torcetrapib; anacetrapid; JTT-705 (Japan Tobacco/Roche); or combinations thereof.

In some embodiments, the modulator of MIF and a fibrate synergistically treat an inflammatory disorder by (1) decreasing the chemotaxis of leukocytes, and (2) increasing the concentration of HDL. In some embodiments, the modulator of MIF also decreases any undesired inflammation resulting from administration of the fibrate.

In some embodiments, the modulator of MIF and an ApoA1 modulator synergistically treat an inflammatory disorder by (1) decreasing the chemotaxis of leukocytes, and (2) increasing the concentration of HDL. In some embodiments, the modulator of MIF also decreases any undesired inflammation resulting from administration of the ApoA1 modulator.

In some embodiments, the modulator of MIF and a CETP modulator synergistically treat an inflammatory disorder by (1) decreasing the chemotaxis of leukocytes, and (2) increasing the concentration of HDL. In some embodiments, the modulator of MIF also decreases any undesired inflammation resulting from administration of the CETP inhibitor.

Pharmaceutical Composition Comprising an Anti-Inflammatory Agent and an Agent that Induces Unwanted Inflammation

Disclosed herein, in certain embodiments, are methods and pharmaceutical compositions for modulating an inflammatory disorder comprising a synergistic combination of (a) a modulator of MIF; and (b) a second active agent that induces unwanted inflammation. In certain instances, administration of the second active agent causes (either partially or fully) undesired inflammation (e.g., pancreatitis). In some embodiments, a higher dose of the second active agent (i.e., a dose that otherwise induces undesired inflammation) is used to treat a disorder because a sufficient dose of the modulator of MIF is co-administered to decrease/manage the undesired inflammation (including pancreatitis) resulting from the administration of the second active agent.

In some embodiments, the modulator of MIF and the second active agent synergistically treat a disorder by (1) the second active agent's treating the disorder and (2) the modulator of MIF's decreasing any undesired inflammation resulting from administration of the second active agent.

In some embodiments, the second active agent is a bisphosphonate. In certain instances, administration of a bisphosphonate causes eye inflammation. Examples of bisphosphonates include, but are not limited to, Etidronate (DIDRONEL®); Clodronate (BONEFOS®); Tiludronate (SKELID®); Pamidronate (APD, AREDIA®); Neridronate; Olpadronate; Alendronate (FOSFAMAX®); Ibandronate (BONIVA®); Risedronate (ACTONEL®); and Zoledronate (ZOMETA®). In some embodiments, the modulator of MIF and a bisphosphonate synergistically treat a bone disorder by (1) decreasing the osteoclast action and the resorption of bone and (2) decreasing any undesired inflammation resulting from administration of the bisphosphonate. In some embodiments, a higher dose of a bisphosphonate (i.e., a dose that otherwise induces eye inflammation) is used to treat a bone disorder because a sufficient dose of the modulator of MIF is also administered to decrease/manage the undesired inflammation (including eye inflammation).

In some embodiments, the second active agent is a protease inhibitor. In certain instances, administration of a protease inhibitor causes liver inflammation. In some embodiments, the modulator of MIF and a protease inhibitor synergistically treat an HIV infection by (1) inhibiting (either partially or fully) the activity of an HIV protease and (2) decreasing any undesired inflammation (including liver inflammation and pancreatitis) resulting from administration of the protease inhibitor. In some embodiments, a higher dose of a protease inhibitor (i.e., a dose that otherwise induces pancreatitis and/or liver inflammation) is used to treat an HIV infection because a sufficient dose of the modulator of MIF is also administered to decrease/manage the undesired inflammation (including pancreatitis and/or liver inflammation). Examples of protease inhibitors include, but are not limited to, Saquinavir (FORTOVASE®, INVIRASE®); Ritonavir (NORVIR®); Indinavir (CRIXIVAN®); Nelfinavir (VIRACEPT®); Amprenavir (AGENERASE®); Lopinavir (KALETRA®); Atazanavir (REYATAZ®); Fosamprenavir (LEXIVA®); Tipranavir (APTIVUS®); Darunavir (PREZISTA®) and Zidovudine (RETROVIR®).

In some embodiments, the second active agent is exenatide. In certain instances, exenatide causes (either partially or fully) pancreatitis. In some embodiments, the modulator of MIF and exenatide synergistically treat diabetes mellitus type 2 by (1) enhancing glucose-dependent insulin secretion by the pancreatic beta-cell and (2) decreasing any undesired inflammation (including pancreatitis) resulting from administration of the second active agent. In some embodiments, a higher dose of exenatide (i.e., a dose that otherwise induces pancreatitis) is used to treat diabetes mellitus type 2 because a sufficient dose of the modulator of MIF is also administered to decrease/manage the undesired inflammation (including pancreatitis).

In some embodiments, the second active agent is ribavirin. In certain instances, administration of ribavirin causes (either partially or fully) pancreatitis. In some embodiments, the modulator of MIF and ribavirin synergistically treat a viral infection by (1) inducing mutations in viruses, inhibiting the activity of RNA polymerases, inhibiting the activity of certain transferase enzymes, and modulating T-cell phenotypes, and (2) decreasing any undesired inflammation (including pancreatitis) resulting from administration of the second active agent. In some embodiments, a higher dose of ribavirin (i.e., a dose that otherwise induces pancreatitis) is used to treat viral infections because a sufficient dose of the modulator of MIF is also administered to decrease/manage the undesired inflammation (including pancreatitis).

In some embodiments, the second active agent is didanosine. In certain instances, administration of didanosine causes (either partially or fully) pancreatitis. In some embodiments, the modulator of MIF and didanosine synergistically treat HIV infections by (1) inhibiting (either partially or fully) the activity of a reverse transcriptase, and (2) decreasing any undesired inflammation (including pancreatitis) resulting from administration of the second active agent. In some embodiments, a higher dose of didanosine (i.e., a dose that otherwise induces pancreatitis) is used to treat HIV infections because a sufficient dose of the modulator of MIF is also administered to decrease/manage the undesired inflammation (including pancreatitis).

In some embodiments, the second active agent is bexarotene. In certain instances, administration of bexarotene causes (either partially or fully) pancreatitis. In some embodiments, the modulator of MIF and bexarotene synergistically treat a proliferative disorder (e.g. cutaneous T cell lymphoma) by (1) regulating cellular differentiation and/or proliferation and (2) decreasing any undesired inflammation (including pancreatitis) resulting from administration of the second active agent. In some embodiments, a higher dose of bexarotene (i.e., a dose that otherwise induces pancreatitis) is used to treat a proliferative disorder because a sufficient dose of the modulator of MIF is also administered to decrease/manage the undesired inflammation (including pancreatitis).

In some embodiments, the second active agent is stavudine. In certain instances, administration of stavudine causes (either partially or fully) pancreatitis. In some embodiments, the modulator of MIF and stavudine synergistically treat an HIV infection by (1) inhibiting (either partially or fully) the activity of a reverse transcriptase and (2) decreasing any undesired inflammation (including pancreatitis) resulting from administration of the second active agent. In some embodiments, a higher dose of stavudine (i.e., a dose that otherwise induces pancreatitis) is used to treat an HIV infection because a sufficient dose of the modulator of MIF is also administered to decrease/manage the undesired inflammation (including pancreatitis).

In some embodiments, the second active agent is denileukin diftitox. In certain instances, administration of denileukin diftitox causes (either partially or fully) hypersensitivity. In some embodiments, the modulator of MIF and denileukin diftitox synergistically treat a proliferative disorder by (1) killing abnormally proliferating cells, and (2) decreasing any undesired inflammation (including pancreatitis) resulting from administration of the second active agent. In some embodiments, a higher dose of denileukin diftitox (i.e., a dose that otherwise induces hypersensitivity) is used to treat a proliferative disorder because a sufficient dose of the modulator of MIF is also administered to decrease/manage the undesired inflammation (including hypersensitivity).

In some embodiments, the second active agent is abacavir. In certain instances, administration of abacavir causes (either partially or fully) hypersensitivity. In some embodiments, the modulator of MIF and abacavir synergistically treat an HIV infection by (1) inhibiting (either partially or fully) the activity of a reverse transcriptase and (2) decreasing any undesired inflammation (including pancreatitis) resulting from administration of the second active agent. In some embodiments, a higher dose of abacavir (i.e., a dose that otherwise induces hypersensitivity) is used to treat an HIV infection because a sufficient dose of the modulator of MIF is also administered to decrease/manage the undesired inflammation (including hypersensitivity).

In some embodiments, the second active agent is propylthiouracil. In certain instances, administration of propylthiouracil causes (either partially or fully) vasculitis. In some embodiments, the modulator of MIF and propylthiouracil synergistically treat hyperthyroidism by (1) inhibiting (partially or fully) the production of thyroid hormone and (2) decreasing any undesired inflammation (including pancreatitis) resulting from administration of the second active agent. In some embodiments, a higher dose of propylthiouracil (i.e., a dose that otherwise induces vasculitis) is used to treat hyperthyroidism because a sufficient dose of the modulator of MIF is also administered to decrease/manage the undesired inflammation (including vasculitis).

In some embodiments, the second active agent is deferasirox. In certain instances, administration of deferasirox causes (either partially or fully) vasculitis and/or hypersensitivity. In some embodiments, the modulator of MIF and deferasirox synergistically treat iron overload by (1) facilitating the elimination of iron from a body and (2) decreasing any undesired inflammation (including vasculitis) resulting from administration of the second active agent. In some embodiments, a higher dose of deferasirox (i.e., a dose that otherwise induces pancreatitis) is used to treat iron overload because a sufficient dose of the modulator of MIF is also administered to decrease/manage the undesired inflammation (including vasculitis).

### Gene Therapy

In some embodiments, are methods and pharmaceutical compositions for modulating an inflammatory disorder, comprising a combination of (a) a therapeutically-effective amount of (a) a modulator of MIF; and (b) gene therapy.

In some embodiments, the gene therapy comprises modulating the concentration of a lipid and/or lipoprotein (e.g., HDL) in the blood of an individual in need thereof. In some embodiments, modulating the concentration of a lipid and/or lipoprotein (e.g., HDL) in the blood comprises transfecting DNA into an individual in need thereof. In some embodiments, the DNA encodes an Apo A1 gene, an LCAT gene, an LDL gene, an Il-4 gene, an IL-10 gene, an IL-1 ra gene, a Galectin-3gene, or combinations thereof. In some embodiments, the DNA is transfected into a liver cell.

In some embodiments, the DNA is transfected into a liver cell via use of ultrasound. For disclosures of techniques related to transfecting ApoA1 DNA via use of ultrasound see U.S. Pat. No. 7,211,248, which is hereby incorporated by reference for those disclosures.

In some embodiments, an individual is administered a vector engineered to carry the human gene (the “gene vector”). For disclosures of techniques for creating an LDL gene vector see U.S. Pat. No. 6,784,162, which is hereby incorporated by reference for those disclosures. In some embodiments, the gene vector is a retrovirus. In some embodiments, the gene vector is not a retrovirus (e.g. it is an adenovirus; a lentivirus; or a polymeric delivery system such as METAFECTENE, SUPERFECT®, EFFECTENE®, or MIRUS TRANSIT). In certain instances, a retrovirus, adenovirus, or lentivirus will have a mutation such that the virus is rendered incompetent.

In some embodiments, the vector is administered in vivo (i.e., the vector is injected directly into the individual, for example into a liver cell), ex vivo (i.e., cells from the individual are grown in vitro and transduced with the gene vector, embedded in a carrier, and then implanted in the individual), or a combination thereof.

In certain instances, after administration of the gene vector, the gene vector infects the cells at the site of administration (e.g. the liver). In certain instances the gene sequence is incorporated into the subject's genome (e.g. when the gene vector is a retrovirus). In certain instances therapy will need to be periodically re-administered (e.g. when the gene vector is not a retrovirus). In some embodiments, therapy is re-administered annually. In some embodiments, therapy is re-administered semi-annually. In some embodiments, therapy is re-administered when the subject's HDL level decreases below about 60 mg/dL. In some embodiments, therapy is re-administered when the subject's HDL level decreases below about 50 mg/dL. In some embodiments, therapy is re-administered when the subject's HDL level decreases below about 45 mg/dL. In some embodiments, therapy is re-administered when the subject's HDL level decreases below about 40 mg/dL. In some embodiments, therapy is re-administered when the subject's HDL level decreases below about 35 mg/dL. In some embodiments, therapy is re-administered when the subject's HDL level decreases below about 30 mg/dL.

### RNAi Therapies

In some embodiments, are methods and pharmaceutical compositions for modulating an inflammatory disorder, comprising a combination of (a) a therapeutically-effective amount of (a) a modulator of MIF; and (b) silencing the expression of a gene that participates in the development and/or progression of an inflammatory disorder (the “target gene”). In some embodiments, the target gene is Apolipoprotein B (Apo B), Heat Shock Protein 110 (Hsp 110), Proprotein Convertase Subtilisin Kexin 9 (Pcsk9), CyD1, TNF-α, IL-β, Atrial Natriuretic Peptide Receptor A (NPRA), GATA-3, Syk, VEGF, MIP1, FasL, DDR-1, C5aR, AP-1, or combinations thereof.

In some embodiments, the target gene is silenced by RNA interference (RNAi). In some embodiments, the RNAi therapy comprises use of a siRNA molecule. In some embodiments, a double stranded RNA (dsRNA) molecule with sequences complementary to an mRNA sequence of a gene to be silenced (e.g., Apo B, Hsp 110 and Pcsk9) is generated (e.g by PCR). In some embodiments, a 20-25 bp siRNA molecule with sequences complementary to an mRNA sequence of a gene to be silenced is generated. In some embodiments, the 20-25 bp siRNA molecule has 2-5 bp overhangs on the 3′ end of each strand, and a 5′ phosphate terminus and a 3′ hydroxyl terminus. In some embodiments, the 20-25 bp siRNA molecule has blunt ends. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, a siRNA molecule is “fully complementary” (i.e., 100% complementary) to the target gene. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target gene. In some embodiments, there is a 1 bp mismatch, a 2 bp mismatch, a 3 bp mismatch, a 4 bp mismatch, or a 5 bp mismatch.

In certain instances, after administration of the dsRNA or siRNA molecule, cells at the site of administration (e.g. the cells of the liver and/or small intestine) are transformed with the dsRNA or siRNA molecule. In certain instances following transformation, the dsRNA molecule is cleaved into multiple fragments of about 20-25 bp to yield siRNA molecules. In certain instances, the fragments have about 2 bp overhangs on the 3′ end of each strand.

In certain instances, a siRNA molecule is divided into two strands (the guide strand and the anti-guide strand) by an RNA-induced Silencing Complex (RISC). In certain instances, the guide strand is incorporated into the catalytic component of the RISC (i.e. argonaute). In certain instances, the guide strand binds to a complementary RB1 mRNA sequence. In certain instances, the RISC cleaves an mRNA sequence of a gene to be silenced. In certain instances, the expression of the gene to be silenced is down-regulated.

In some embodiments, a sequence complementary to an mRNA sequence of a target gene is incorporated into a vector. In some embodiments, the sequence is placed between two promoters. In some embodiments, the promoters are orientated in opposite directions. In some embodiments, the vector is contacted with a cell. In certain instances, a cell is transformed with the vector. In certain instances following transformation, sense and anti-sense strands of the sequence are generated. In certain instances, the sense and anti-sense strands hybridize to form a dsRNA molecule which is cleaved into siRNA molecules. In certain instances, the strands hybridize to form a siRNA molecule. In some embodiments, the vector is a plasmid (e.g pSUPER; pSUPER.neo; pSUPER.neo+gfp).

In some embodiments, a siRNA molecule is administered to in vivo (i.e., the vector is injected directly into the individual, for example into a liver cell or a cell of the small intestine, or into the blood stream).

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering an Nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, a siRNA molecule described herein is administered to the liver by any suitable manner. For methods of administering an antisense molecule described herein see Wen et al., 2004, World J Gastroenterol., 10, 244-9; Murao et al., 2002, Pharm Res., 19, 1808-14; Liu et al., 2003, Gene Ther., 10, 180-7; Hong et al., 2003, J Pharm Pharmacol., 54, 51-8; Herrmann et al., 2004, Arch Virol., 149, 1611-7; and Matsuno et al., 2003, Gene Ther., 10, 1559-66) all of which are hereby incorporated by reference for such disclosures.

In some embodiments, a siRNA molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, a siRNA molecule described herein is administered systemically (i.e., in vivo systemic absorption or accumulation of a siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances therapy will need to be periodically re-administered. In some embodiments, therapy is re-administered annually. In some embodiments, therapy is re-administered semi-annually. In some embodiments, therapy is administered monthly. In some embodiments, therapy is administered weekly.

For disclosures of techniques related to silencing the expression of Apo B and/or Hsp110 see U.S. Pub. No. 2007/0293451 which is hereby incorporated by reference for such disclosures. For disclosures of techniques related to silencing the expression of Pcsk9 see U.S. Pub. No. 2007/0173473 which is hereby incorporated by reference for such disclosures.

### Antisense Therapies

In some embodiments, are methods and pharmaceutical compositions for modulating an inflammatory disorder, comprising a combination of (a) a therapeutically-effective amount of (a) a modulator of MIF; and (b) inhibiting the expression of and/or activity of a DNA or RNA sequence that participates in the development and/or progression of an inflammatory disorder (the “target sequence”). In some embodiments, inhibiting the expression of and/or activity of a target sequence comprises use of an antisense molecule complementary to the target sequence. In some embodiments, the target sequence is microRNA-122 (miRNA-122 or mRNA-122), secretory phospholipase A2 (sPLA2), intracellular adhesion molecule-1 (ICAM-1), GATA-3, NF-κ B, Syk, or combinations thereof. In certain instances, inhibiting the expression of and/or activity of miRNA-122 results (partially or fully) in a decrease in the concentration of cholesterol and/or lipids in blood.

In some embodiments, an antisense molecule that is complementary to a target sequence is generated (e.g. by PCR). In some embodiments, the antisense molecule is about 15 to about 30 nucleotides. In some embodiments, the antisense molecule is about 17 to about 28 nucleotides. In some embodiments, the antisense molecule is about 19 to about 26 nucleotides. In some embodiments, the antisense molecule is about 21 to about 24 nucleotides. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, the antisense molecules are single-stranded, double-stranded, circular or hairpin. In some embodiments, the antisense molecules contain structural elements (e.g., internal or terminal bulges, or loops).

In some embodiments, an antisense molecule is “fully complementary” (i.e., 100% complementary) to the target sequence. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target RNA sequence. In some embodiments, there is a 1 bp mismatch, a 2 bp mismatch, a 3 bp mismatch, a 4 bp mismatch, or a 5 bp mismatch.

In some embodiments, the antisense molecule hybridizes to the target sequence. As used herein, “hybridize” means the pairing of nucleotides of an antisense molecule with corresponding nucleotides of the target sequence. In certain instances, hybridization involves the formation of one or more hydrogen bonds (e.g., Watson-Crick, Hoogsteen or reversed Hoogsteen hydrogen bonding) between the pairing nucleotides.

In certain instances, hybridizing results (partially or fully) in the degradation, cleavage, and/or sequestration of the RNA sequence.

In some embodiments, the antisense molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering an Nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, the antisense molecule described herein is administered to the liver by any suitable manner. For methods of administering an antisense molecule described herein see Wen et al., 2004, World J Gastroenterol., 10, 244-9; Murao et al., 2002, Pharm Res., 19, 1808-14; Liu et al., 2003, Gene Ther., 10, 180-7; Hong et al., 2003, J Pharm Pharmacol., 54, 51-8; Herrmann et al., 2004, Arch Virol., 149, 1611-7; and Matsuno et al., 2003, Gene Ther., 10, 1559-66) all of which are hereby incorporated by reference for such disclosures.

In some embodiments, the antisense molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, the antisense molecule described herein is administered systemically (i.e., in vivo systemic absorption or accumulation of a siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances therapy will need to be periodically re-administered. In some embodiments, therapy is re-administered annually. In some embodiments, therapy is re-administered semi-annually. In some embodiments, therapy is administered monthly. In some embodiments, therapy is administered weekly.

For disclosures of techniques related to silencing the expression of miRNA-122 see WO 07/027,775A2 which is hereby incorporated by reference for such disclosures.

### VII. PHARMACEUTICAL COMPOSITIONS

Disclosed herein, in certain embodiments, is a pharmaceutical composition for modulating an inflammation, comprising a synergistic combination of (a) a therapeutically-effective amount of a modulator of MIF; and (b) a therapeutically-effective amount of a second active agent selected from a modulator of a lipid disorder.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition for modulating an inflammation further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions described herein are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, modified release formulations, delayed release formulations, extended release formulations, pulsatile release formulations, multiparticulate formulations, and mixed immediate release and controlled release formulations.

### Multiparticulate Formulations

In some embodiments, the pharmaceutical compositions described herein are formulated as mulitparticulate formulations. In some embodiments, the pharmaceutical compositions described herein comprise a first population of particles and a second population of particles. In some embodiments, the first population comprises an active agent. In some embodiments, the second population comprises an active agent. In some embodiments, the dose of active agent in the first population is equal to the dose of active agent in the second population. In some embodiments, the dose of active agent in the first population is not equal to (e.g., greater than or less than) the dose of active agent in the second population.

In some embodiments, the active agent of the first population is released before the active agent of the second population. In some embodiments, the second population of particles comprises a modified-release (e.g., delayed-release, controlled-release, or extended release) coating. In some embodiments, the second population of particles comprises a modified-release (e.g., delayed-release, controlled-release, or extended release) matrix.

Coating materials for use with the pharmaceutical compositions described herein include, but are not limited to, polymer coating materials (e.g., cellulose acetate phthalate, cellulose acetate trimaletate, hydroxy propyl methylcellulose phthalate, polyvinyl acetate phthalate); ammonio methacrylate copolymers (e.g., Eudragit® RS and RL); poly acrylic acid and poly acrylate and methacrylate copolymers (e.g., Eudragite S and L, polyvinyl acetaldiethylamino acetate, hydroxypropyl methylcellulose acetate succinate, shellac); hydrogels and gel-forming materials (e.g., carboxyvinyl polymers, sodium alginate, sodium carmellose, calcium carmellose, sodium carboxymethyl starch, poly vinyl alcohol, hydroxyethyl cellulose, methyl cellulose, gelatin, starch, hydroxypropyl cellulose, hydroxypropyl methylcellulose, polyvinylpyrrolidone, crosslinked starch, microcrystalline cellulose, chitin, aminoacryl-methacrylate copolymer, pullulan, collagen, casein, agar, gum arabic, sodium carboxymethyl cellulose, (swellable hydrophilic polymers) poly(hydroxyalkyl methacrylate) (MW about 5 k-about 5,000 k), polyvinylpyrrolidone (MW about 10 k-about 360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (MW about 30 k-about 300 k), polysaccharides such as agar, acacia, karaya, tragacanth, algins and guar, polyacrylamides, Polyox® polyethylene oxides (MW about 100 k-about 5,000 k), AquaKeep® acrylate polymers, diesters of polyglucan, crosslinked polyvinyl alcohol and poly N-vinyl-2-pyrrolidone, sodium starch; hydrophilic polymers (e.g., polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, other acrylic acid derivatives, sorbitan esters, natural gums, lecithins, pectin, alginates, ammonia alginate, sodium, calcium, potassium alginates, propylene glycol alginate, agar, arabic gum, karaya gum, locust bean gum, tragacanth gum, carrageens gum, guar gum, xanthan gum, scleroglucan gum); or combinations thereof. In some embodiments, the coating comprises a plasticiser, a lubricant, a solvent, or combinations thereof. Suitable plasticisers include, but are not limited to, acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

In some embodiments, the second population of particles comprises a modified release matrix material. Materials for use with the pharmaceutical compositions described herein include, but are not limited to microcrystalline cellulose, sodium carboxymethylcellulose, hydroxyalkylcelluloses (e.g., hydroxypropylmethylcellulose and hydroxypropylcellulose), polyethylene oxide, alkylcelluloses (e.g., methylcellulose and ethylcellulose), polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate, or combinations thereof.

### Other Formulations

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations disclosed herein are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a MIF receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical compositions disclosed herein are optionally in a form of an aerosol, a mist or a powder. Pharmaceutical compositions described herein are conveniently delivered in the form of an aerosol spray presentation from pressurized packs or an Nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix and a suitable powder base such as lactose or starch.

Buccal formulations include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of the pharmaceutical compositions disclosed herein are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) an active agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an active agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an active agent optionally with carriers, optionally a rate controlling barrier to deliver a an active agent to the skin of the host at a controlled and predetermined rate over a prolonged period of time, and means to secure the device to the skin.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, an active agent disclosed herein is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

An active agent disclosed herein is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### VIII. DOSAGES AND ADMINISTRATION

In some embodiments, the pharmaceutical compositions disclosed herein are administered to an individual in need thereof. In some embodiments, the pharmaceutical compositions disclosed herein are administered to an individual diagnosed with (i.e., satisfies the diagnostic criteria for) an inflammatory disorder (e.g. rheumatoid arthritis, SLE or cancer). In some embodiments, the pharmaceutical compositions disclosed herein are administered to an individual suspected of having an inflammatory disorder. In some embodiments, the pharmaceutical compositions disclosed herein are administered to an individual predisposed to develop an inflammatory disorder.

In certain instances, an individual is at risk of inflammatory bowel disease if elevated levels of bacterial antigens I2, OmpC or flagellin are present in the serum. In certain instances, an individual is at risk of Crohn's disease if perinuclear antineutrophil cytoplasmic antigens are detected in the serum. In certain instances, an individual is at risk of rheumatoid arthritis if the expression of IL-1β and its type II receptor is significantly upregulated in the blood. In certain instances, an individual is at risk of rheumatoid arthritis if the IL-6 levels are elevated in blood. In certain instances, an individual is at risk of SLE if MicroRNA 95 (miR 95) expression is one third of the gene expression of the microRNA 95 of controls. In certain instances, an individual is at risk of B-cell lymphoma if CD40 expression is upregulated on B cells. In certain instances, an individual is at risk of prostate cancer if PSA levels are elevated in blood.

The daily dosages appropriate for an active agent disclosed herein are from about 0.01 to 3 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a Number of variables, not limited to the activity of the active agents used, the diseases or conditions to be treated, the mode of administration, the requirements of the individual subject, the severity of the disease or condition being treated, and the judgment of the practitioner.

In some embodiments, administration of the lipid modulating agent results in (either partially or fully) undesired inflammation. In some embodiments, administration of the second anti-inflammatory agent results in (either partially or fully) undesired inflammation. In some embodiments, the first anti-inflammatory agent is administered to the individual to treat the undesired inflammation from the second anti-inflammatory agent or the lipid modulating agent. In some embodiments, the administration of the second anti-inflammatory agent or lipid modulating agent is discontinued until the inflamed cells and/or tissue are no longer inflamed. In some embodiments, after the inflamed cells and/or tissue are no longer inflamed, administration of the second inflammatory agent or lipid modulating agent recommences. In some embodiments, administration of the second anti-inflammatory agent or lipid modulating agent recommences in combination with an alternative dose of the first anti-inflammatory agent.

In the case wherein the individual's condition does not improve, upon the doctor's discretion the administration of an active agent disclosed herein is optionally administered chronically, that is, for an extended period of time, including throughout the duration of the individual's life in order to ameliorate or otherwise control or limit the symptoms of the individual's disease or condition.

In the case wherein the individual's status does improve, upon the doctor's discretion the administration of an active agent disclosed herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is therapeutic index, which is expressed as the ratio between LD50 and ED50. An active agent disclosed herein exhibiting high therapeutic indices is preferred. The data obtained from cell culture assays and animal studies are optionally used in formulating a range of dosage for use in human. The dosage of such an active agent disclosed herein lies preferably within a range of circulating concentrations that include the ED50 with minimal toxicity. The dosage optionally varies within this range depending upon the dosage form employed and the route of administration utilized.

In some embodiments, methods disclosed herein are used before, during, and/or after an organ transplant. In some embodiments, compositions disclosed herein are administered before, during, and/or after an organ transplant. In certain instances, an “inflammatory/cytokine storm” develops following an organ transplant. In some embodiments, the organ transplant is a heart, kidney, lung transplant. In certain instances, an inflammatory cytokine storm comprises high fever, swelling and redness, extreme fatigue, and nausea. In some embodiments, a modulator of MIF is administered in combination with cyclosporin A.

### EXAMPLES

### Example 1

### Preparation of a Multi-Particulate Dosage Form

10 kg Methotrexate is first screened through a suitable screen (e.g. 500 micron). 25 kg Lactose monohydrate, 8 kg hydroxypropylmethyl cellulose, the screened methotrexate and 5 kg calcium hydrogen phosphate (anhydrous) are then added to a suitable blender (e.g. a tumble mixer) and blended. The blend is screened through a suitable screen (e.g. 500 micron) and reblended. About 50% of the lubricant (2.5 kg, magnesium stearate) is screened, added to the blend and blended briefly.

The blend is roller compacted through a suitable roller compactor. The ribbon blend is then granulated, by screening through a suitable screen (e.g. 500 micron) and reblended. The remaining lubricant (2 kg, magnesium stearate) is screened, added to the blend and blended briefly. The granules are screened (e.g. 200 micron) to obtain granulation particles of the desired size.

GAQNRSYSKLLCG (hereinafter, Peptide-1) granules are prepared by blending 2.8 kg Peptide 1 with microcrystalline cellulose (Avicel® PH101, FMC Corp., Philadelphia, Pa.) in relative amounts of 95:5 (w/w), wet massing the blend in a Hobart mixer with water equivalent to approximately 27% of the weight of the blend, extruding the wet mass through a perforated plate (Luwa EXKS-1 extruder, Fuji Paudal Co., Osaka Japan), spheronizing the extrudate (Luwa QJ-230 marumerizer, Fuji Paudal Co.) and drying the final granules which are about 1 mm diameter. The granules are optionally coated with a plasticized ethylcellulose dispersion (Surelease®, Colorcon, West Point, Pa., typically applied at 15% solids concentration) in a bottom spray Wurster fluid bed coater (Aeromatic Strea-1, Niro Inc., Bubendorf, Switzerland) to obtain sustained release granules. The amount of coating applied is varied to obtain different dissolution rate behavior. For example, an additional coating of 2% Opadry® is optionally applied over the Surelease® Coat.

The methotrexate immediate release granules and the peptide 1 sustained release granules are mixed together and the resulting mixture is encapsulated in gelatin capsules.

### Example 2

### In vivo Investigations in a Rat Model of Arthritis Disease to Test Combination of Etanercept and a Modulator of MIF

31 Male Lewis rats are immunized with complete Freund's adjuvant on day 0 to induce an aggressive arthritis characterized by joint destruction and paw swelling.

From day 8 to 20, two groups of rats receive thrice weekly intraperitoneal injections of 50 μg of GAQNRSYSKLLCG (hereinafter, Peptide 1) (n=12 rats). During this time, the two groups of rats also receive weekly subcutaneous injections of 50 g Etanercept. An untreated group of rats (n=12) serve as a control.

Every week, paw swelling is determined by water displacement plethysmometry. The extent of arthritis is determined at the end of the study on day 21. Radiographs are obtained of the right hind paw to assess bone changes using a semi-quantitative scoring system: demineralization (0-2+), calcaneal erosion (0-1+), and heterotropic bone formation (0-1+), with a maximum possible score=6. Blood samples are tested for neutropenia.

### Example 3

### In vivo Investigations in a Rat Model of Crohn's Disease to Test Combination of Methotrexate and a Modulator of MIF

A modified animal model disclosed in Kirkil, C. et al., J Gastrointest Surg. 2008, 12, 1429-35 is used. Twenty-eight Sprague-Dawley rats are divided into four groups. Groups I and II are used as sham-operated and control groups, respectively. Bowel inflammation is induced by intrajejunal injection of iodoacetamide in groups III and IV. Rats in group IV are treated with oral preparation of methotrexate (10 mg) and intravenous injection of 50 μg of GAQNRSYSKLLCG (hereinafter, Peptide 1) (n=12 rats).

Three days after induction of the inflammation, partial resection of test loop and anastomosis is performed. Re-laparotomy is performed, anastomosis bursting pressures and peritonitis scores are measured, and tissue samples are obtained for the measurements of tissue hydroxylproline level and mucosal damage index 4 days later.

On the fourth day, measurements of tissue hydroxylproline level and mucosal damage index are obtained. The severity of iodoacetamide induced intestinal inflammation, wound healing in the inflamed intestinal tissue, and decrease in severity of peritonitis is also recorded.

### Example 4

### Human Clinical Trial in SLE to Test Combination of Cyclophosphamide and a Modulator of MIF

Study Objective(s): The primary objective of this study is to assess efficacy of the fixed combination cyclophosphamide/GAQNRSYSKLLCG (hereinafter, Peptide 1, or P1) (C/P1; 60/20 mg, 60/40 mg, 60/80 mg) in subjects with systemic lupus erythematosus (SLE) who are currently receiving cyclophosphamide. This study will also determine if P1 is effective in decreasing disease activity in these patients.

### Methods

The first part of the study is a dose-escalation study in which participants will receive one of two doses of P1 (20 mg, or 40 mg); this part of the study will last 60 days. At screening, patients will have an IV catheter inserted into their arms for administration of cyclophosphamide and P1. Patients will also have medical and medication history assessments, a comprehensive physical exam, and blood and urine tests. There are 5 study visits for the first part of the trial; these will occur at screening, at study entry, and Days 1, 14, and 28. Selected visits will include physical exam, vital signs measurement, blood and urine tests, and disease activity assessment. At Days 7 and 60, patients will be contacted by phone to report their medication history and any adverse effects they have experienced.

The second part of the study will evaluate a single 80 mg dose of P1; this part of the study will last 90 days. In the study, participants will be randomly assigned to one of two groups. At the start of the study, Group 1 participants will receive P1 and cyclophosphamide and Group 2 participants will receive cyclophosphamide only. There will be 9 study visits; these will occur at study screening, study entry, and Days 1, 4, 7, 14, 28, and 60. At selected visits, patients will undergo physical exam, vital signs measurement, blood tests and urine tests, and disease activity assessment.

Number of Subjects: It is planned to recruit between 30 and 50 subjects for each part of the study.

Diagnosis and Main Criteria for Inclusion: Diagnosis of SLE by American College of Rheumatology (ACR) criteria

Concurrent treatment with intravenous cyclophosphamide for at least one of the following manifestations of lupus: World Health Organization (WHO) class III, IV, or V lupus nephritis; British Isles Lupus Assessment Group (BILAG) score of A for vasculitis; BILAG score of A for cytopenia; BILAG score of A for nervous system; Stable medication regimen for at least 4 weeks prior to study entry; Weight between 40 kg (88.2 lbs) and 125 kg (275.6 lb).

Study Treatment: During the study periods, subjects will have an IV catheter inserted into their arms for intravenous bi-weekly administration of cyclophosphamide and P1.

Efficacy Evaluations: The primary endpoint is SLE disease activity as measured by blood tests, urine tests, and disease activity assessment.

Safety Evaluations: Safety is assessed using routine clinical laboratory evaluations (lupus serology and renal function).

### Example 5

### Human Clinical Trial in Rheumatoid Arthritis to Test Combination of Infliximab and a Modulator of MIF

Study Objective(s): The primary objective of this study is to assess efficacy of the fixed combination infliximab/GAQNRSYSKLLCG (hereinafter, Peptide 1 or P1) (I/P1; 5 mg/kg/20 mg, 10 mg/kg/20 mg, 15 mg/kg/20 mg) in subjects with rheumatoid arthritis who are currently receiving infliximab for treatment of rheumatoid arthritis. This study will also determine if P1 is effective in decreasing disease activity in these patients.

### Methods

Participants will receive nine infusions of infliximab and P1 every three weeks during this 28-week study. The drug is given intravenously (IV, into a vein) over 2 hours. The first three infusions will be at a dose of 5 mg/kg of body weight. Patients will also receive 20 mg P1 in a saline solution (IV, into a vein) over 1 hour. Patients who improve on this regimen will receive another 6 infusions at the same dose. Patients who do not significantly improve on 5 mg/kg at the end of 6 weeks (the third infusion) may continue with phase 2 of the study, in which they will be randomly assigned to receive either: 1) 6 additional doses of tinfliximab at 5 mg/kg per dose, or 2) a gradually increased dose of inflilximab to a maximum of 15 mg/kg. In addition, all patients will continue to take P1 at the same dose as when they entered the study.

Patients will have imaging studies (x-rays, MRI and Dexa scan) at the beginning and end of the study and will collect a 24-hour urine sample before each infliximab and P1 infusion.

Number of Subjects: It is planned to recruit between 30 and 50 subjects for each part of the study.

Inclusion criteria: Patients must be at least 18 years old at the screening visit. Patients must have a diagnosis of adult-onset RA of at least six months duration but not longer than fifteen years as defined by the 1987 American College of Rheumatology classification criteria.

Patients must have active RA disease as defined by: 9 tender joints at Screening and Baseline, 9 swollen joints at Screening and Baseline. and fulfilling 1 of the following 2 criteria during the screening period, 30 mm/hour ESR (Westergren), or CRP>15 mg/L.

Patients must have received treatment with infliximab for at least 6 months prior to the Baseline visit. The dose of infliximab and route of administration must have been stable for at least 2 months prior to the baseline visit. The minimum stable dose of infliximab allowed is 5 mg/kg weekly.

Exclusion criteria: Patients must not have a diagnosis of any other inflammatory arthritis (e.g., psoriatic arthritis or ankylosing spondylitis), Patients must not have a secondary, non-inflammatory type of arthritis (e.g. OA or fibromyalgia), Female patients who are breast feeding, pregnant, or plan to become pregnant during the trial or for three months following last dose of study drug, Patients with a history of tuberculosis or positive chest X-ray for tuberculosis or positive, Patients at a high risk of infection (e.g. leg ulcers, indwelling urinary catheter and persistent or recurrent chest infections and patients who are permanently bed ridden or wheelchair bound), Patients with known human immunodeficiency virus (HIV) infection, Patients with an active malignancy of any type or a history of malignancy (except basal cell carcinoma of the skin that has been excised prior to study start), Patients with a current or recent history, as determined by the Investigator, of severe, progressive, and/or uncontrolled renal, hepatic, hematological, gastrointestinal, endocrine, pulmonary, cardiac, neurological, or cerebral disease which would interfere with the patient's participation in the trial, Patients with a history of, or suspected, demyelinating disease of the central nervous system (e.g. multiple sclerosis or optic neuritis).

Primary Outcome measures: Compare efficacy of two dose regimens of infliximab in combination with P1 to infliximab alone in patients with RA measured by the ACR20 at week 28.

Secondary outcome measures: Assess Safety and Tolerability of two dose regimens of infliximab in combination with P1 and infliximab alone in patients with RA; prevention of joint damage in patients with RA; Health Outcomes Measures

Study treatment: During the study periods, subjects will have an IV catheter inserted into their arms for intravenous administration of infliximab and P1.

Efficacy evaluations: The primary endpoint is rheumatoid arthritis disease activity as measured by blood tests, urine tests, x-rays and disease activity assessment.

Safety Evaluations: Safety is assessed using routine clinical laboratory evaluations (blood tests, urine tests).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating an inflammatory disorder, comprising co-administering to an individual in need thereof a synergistic combination of (a) a therapeutically-effective amount of a modulator of MIF selected from: (i) an agent that inhibits MIF binding to CXCR2 and CXCR4 and/or inhibits MIF-activation of CXCR2 and CXCR4; or (ii) an agent that inhibits the ability of MIF to form a homomultimer; and (b) a second active agent selected from an agent that treats an inflammatory disorder.

2. The method of claim 1, wherein the second active agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent an alkylating agent, an anti-metabolite, a plant alkaloid, a terpenoids, a topoisomerase inhibitor, an antitumour antibiotic, a antibody, a hormonal therapy, an anti-diabetes agent, a leukotriene inhibitor, or a combination thereof.

3. The method of claim 1, wherein second active agent is selected from alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), ), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-C5 antibody (e.g., eculizumab); doxycycline; or combinations thereof.

4. The method of claim 1, wherein the second active agent is administered before, after, or simultaneously with the modulator of inflammation.

5. The method of claim 1, wherein the disorder is Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjë gren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease; abdominal aortic aneurysm (AAA); or combinations thereof.

6. A pharmaceutical composition for modulating an inflammatory disorder, comprising a synergistic combination of (a) a therapeutically-effective amount of an agent that treats an inflammatory disorder; and (b) a therapeutically-effective amount of a modulator of MIF selected from: (i) an agent that inhibits MIF binding to CXCR2 and CXCR4 and/or inhibits MIF-activation of CXCR2 and CXCR4; or (ii) an agent that inhibits the ability of MIF to form a homomultimer.

7. The composition of claim 6, wherein the second active agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent an alkylating agent, an anti-metabolite, a plant alkaloid, a terpenoids, a topoisomerase inhibitor, an antitumour antibiotic, a antibody, a hormonal therapy, an anti-diabetes agent, a leukotriene inhibitor, or a combination thereof.

8. The composition of claim 6, wherein the second active agent is selected from alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), ), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S—[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-C5 antibody (e.g., eculizumab; doxycycline; or combinations thereof.

9. The composition of claim 6, wherein the composition comprises a first population of particles and a second population of particles.

10. The composition of claim 9, wherein the first population of particles is formulated for immediate release.

11. The composition of claim 9, wherein the second population of particles is formulated for controlled release.

12. The composition of claim 9, wherein the first population of particles comprises a therapeutically-effective amount of an agent that treats an inflammatory disorder.

13. The composition of claim 9, wherein the second population of particles comprises a therapeutically-effective amount of a modulator of MIF.
